(19) United States
(12) Patent Application Publication
(10) Pub. No.: US 2023/0045419 A1
(43) Pub. Date: Feb. 9, 2023

(54) Solid Forms of 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, 1,3-Dihydroxy-2-(hydroxymethyl)propan-2-amine Salt

(71) Applicant: Pfizer Inc., New York, NY (US)

(72) Inventors: Gary E. Aspnes, Biberach an der Riss (DE); Scott W. Bagley, Mystic, CT (US); Wesley Dewitt Clark, Gales Ferry, CT (US); John M. Curto, Mystic, CT (US); David James Edmonds, Riehen (CH); Mark E. Flanagan, Gales Ferry, CT (US); Kentaro Futatsugi, Sharon, MA (US); David Andrew Griffith, Sudbury, MA (US); Kim Huard, Berkeley, CA (US); Yajing Lian, Waterford, CT (US); Chris Limberakis, Pawcatuck, CT (US); Allyn T. Londregan, Barrington, RI (US); Alan M. Mathiowetz, Waltham, MA (US); David Walter Piotrowski, Waterford, CT (US); Roger B. Ruggeri, Waterford, CT (US)

(73) Assignee: Pfizer Inc., New York, NY (US)

(21) Appl. No.: 17/782,478

(22) PCT Filed: Dec. 7, 2020
(86) PCT No.: PCT/IB2020/061585

§ 371 Date: Jun. 3, 2022

Related U.S. Application Data
(60) Provisional application No. 62/946,084, filed on Dec. 10, 2019.

(51) Int. Cl. C07D 405/14

(52) U.S. Cl. CPC C07D 405/14; C07B 2200/13

(57) ABSTRACT

The invention provides solid forms of 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, 1,3-dihydroxy-2-(hydroxymethyl)propan-2-amine salt for example, a hydrate (e.g. a monohydrate) crystalline form (e.g. Form 2 or Form 3) or an amorphous form; as well as pharmaceutical compositions, and the uses thereof in treating diseases, conditions or disorders modulated by GLP-1R in a mammal, such as a human.

### FIELD OF INVENTION

The invention provides solid forms (e.g. crystalline forms) of 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, 1,3-dihydroxy-2-(hydroxymethyl)propan-2-amine salt; processes for preparing thereof; pharmaceutical compositions, dosage forms, and uses thereof in treating diseases, conditions or disorders modulated by GLP-1R in a mammal such as a human.

### BACKGROUND OF THE INVENTION

Diabetes is a major public health concern because of its increasing prevalence and associated health risks. The disease is characterized by high levels of blood glucose resulting from defects in insulin production, insulin action, or both. Two major forms of diabetes are recognized, Type 1 and Type 2. Type 1 diabetes (T1 D) develops when the body's immune system destroys pancreatic beta cells, the only cells in the body that make the hormone insulin that regulates blood glucose. To survive, people with Type 1 diabetes must have insulin administered by injection or a pump. Type 2 diabetes mellitus (referred to generally as T2DM) usually begins with either insulin resistance or when there is insufficient production of insulin to maintain an acceptable glucose level.

Currently, various pharmacological approaches are available for treating hyperglycemia and subsequently, T2DM (Hampp, C. et al. Use of Antidiabetic Drugs in the U.S., 2003-2012, Diabetes Care 2014, 37, 1367-1374). These may be grouped into six major classes, each acting through a different primary mechanism: (A) Insulin secretogogues, including sulphonyl-ureas (e.g., glipizide, glimepiride, glyburide), meglitinides (e.g., nateglidine, repaglinide), dipeptidyl peptidase IV (DPP-IV) inhibitors (e.g., sitagliptin, vildagliptin, alogliptin, dutogliptin, linagliptin, saxogliptin), and glucagon-like peptide-1 receptor (GLP-1R) agonists (e.g., liraglutide, albiglutide, exenatide, lixisenatide, dulaglutide, semaglutide), which enhance secretion of insulin by acting on the pancreatic beta-cells. Sulphonyl-ureas and meglitinides have limited efficacy and tolerability, cause weight gain and often induce hypoglycemia. DPP-IV inhibitors have limited efficacy. Marketed GLP-1R agonists are peptides administered by subcutaneous injection. Liraglutide is additionally approved for the treatment of obesity. (B) Biguanides (e.g., metformin) are thought to act primarily by decreasing hepatic glucose production. Biguanides often cause gastrointestinal disturbances and lactic acidosis, further limiting their use. (C) Inhibitors of alpha-glucosidase (e.g., acarbose) decrease intestinal glucose absorption. These agents often cause gastrointestinal disturbances. (D) Thiazolidinediones (e.g., pioglitazone, rosiglitazone) act on a specific receptor (peroxisome proliferator-activated receptor-gamma) in the liver, muscle and fat tissues. They regulate lipid metabolism subsequently enhancing the response of these tissues to the actions of insulin. Frequent use of these drugs may lead to weight gain and may induce edema and anemia. (E) Insulin is used in more severe cases, either alone or in combination with the above agents, and frequent use may also lead to weight gain and carries a risk of hypoglycemia. (F) sodium-glucose linked transporter cotransporter 2 (SGLT2) inhibitors (e.g., dapagliflozin, empagliflozin, canagliflozin, ertugliflozin) inhibit reabsorption of glucose in the kidneys and thereby lower glucose levels in the blood. This emerging class of drugs may be associated with ketoacidosis and urinary tract infections.

However, with the exception of GLP-1R agonists and SGLT2 inhibitors, the drugs have limited efficacy and do not address the most important problems, the declining β-cell function and the associated obesity.

Obesity is a chronic disease that is highly prevalent in modern society and is associated with numerous medical problems including hypertension, hypercholesterolemia, and coronary heart disease. It is further highly correlated with T2DM and insulin resistance, the latter of which is generally accompanied by hyperinsulinemia or hyperglycemia, or both. In addition, T2DM is associated with a two to fourfold increased risk of coronary artery disease. Presently, the only treatment that eliminates obesity with high efficacy is bariatric surgery, but this treatment is costly and risky. Pharmacological intervention is generally less efficacious and associated with side effects. There is therefore an obvious need for more efficacious pharmacological intervention with fewer side effects and convenient administration.

Although T2DM is most commonly associated with hyperglycemia and insulin resistance, other diseases associated with T2DM include hepatic insulin resistance, impaired glucose tolerance, diabetic neuropathy, diabetic nephropathy, diabetic retinopathy, obesity, dyslipidemia, hypertension, hyperinsulinemia, and nonalcoholic fatty liver disease (NAFLD).

NAFLD is the hepatic manifestation of metabolic syndrome, and is a spectrum of hepatic conditions encompassing steatosis, non-alcoholic steatohepatitis (NASH), fibrosis, cirrhosis and ultimately hepatocellular carcinoma. NAFLD and NASH are considered the primary fatty liver diseases as they account for the greatest proportion of individuals with elevated hepatic lipids. The severity of NAFLD/NASH is based on the presence of lipid, inflammatory cell infiltrate, hepatocyte ballooning, and the degree of fibrosis. Although not all individuals with steatosis progress to NASH, a substantial portion does.

GLP-1 is a 30 amino acid long incretin hormone secreted by the L-cells in the intestine in response to ingestion of food. GLP-1 has been shown to stimulate insulin secretion in a physiological and glucose-dependent manner, decrease glucagon secretion, inhibit gastric emptying, decrease appetite, and stimulate proliferation of beta-cells. In non-clinical experiments GLP-1 promotes continued beta-cell competence by stimulating transcription of genes important for glucose-dependent insulin secretion and by promoting beta-cell neogenesis (Meier, et al. Biodrugs. 2003; 17 (2): 93-102).

In a healthy individual, GLP-1 plays an important role regulating post-prandial blood glucose levels by stimulating glucose-dependent insulin secretion by the pancreas resulting in increased glucose absorption in the periphery. GLP-1 also suppresses glucagon secretion, leading to reduced hepatic glucose output. In addition, GLP-1 delays gastric emptying and slows small bowel motility delaying food absorption. In people with T2DM, the normal post-prandial rise in GLP-1 is absent or reduced (Vilsboll T, et al. Diabetes. 2001. 50; 609-613).

Holst (Physiol. Rev. 2007, 87, 1409) and Meier (Nat. Rev. Endocrinol. 2012, 8, 728) describe that GLP-1 receptor agonists, such as GLP-1, liraglutide and exendin-4, have 3 major pharmacological activities to improve glycemic control in patients with T2DM by reducing fasting and postprandial glucose (FPG and PPG): (i) increased glucose-dependent insulin secretion (improved first- and second-phase), (ii) glucagon suppressing activity under hyperglycemic conditions, (iii) delay of gastric emptying rate resulting in retarded absorption of meal-derived glucose.

There remains a need for an easily-administered prevention and/or treatment for cardiometabolic and associated diseases.

2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid (referred to herein as “Compound 1”) is a GLP1 agonist.

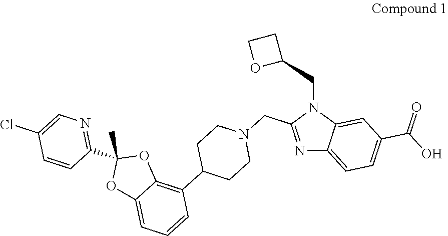 [SMILES: C[C@]1(c2ccc(Cl)cn2)Oc2cccc(C3CCN(Cc4nc5ccc(C(=O)O)cc5n4C[C@@H]4CCO4)CC3)c2O1]

Compound 1 (both in the forms the free acid and as its tris salt) was prepared in Example 10 of U.S. patent application Ser. No. 16/436,311 filed Jun. 10, 2019 and of International Application No. PCT/IB2019/054867 filed Jun. 11, 2019, each of which is hereby incorporated herein by reference in its entirety. There, Compound 1 was designated as 2-({4-[2-(5-chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2:

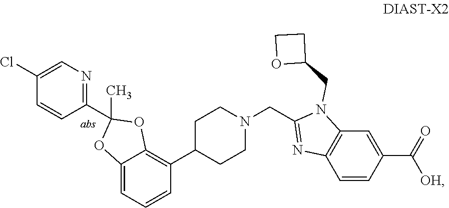 [SMILES: CC1(c2ccc(Cl)cn2)Oc2cccc(C3CCN(Cc4nc5ccc(C(=O)O)cc5n4C[C@@H]4CCO4)CC3)c2O1]

wherein the chiral center on the left part of the compound structure is marked as “abs” to indicate that chiral center has only one stereo-configuration (i.e., not a racemate with respect to that chiral center).

In addition, U.S. patent application Ser. No. 16/436,311 and International Application No. PCT/IB2019/054867 reported an anhydrous crystalline form (designed as Form A) of the tris salt of Compound 1.

It is well known that a solid form, for example a crystalline form of a particular drug (including, e.g., anhydrate, hydrate, solvate, etc.) is often an important determinant of the drug's ease of preparation, stability, solubility, storage stability, ease of formulation, ease of handling, and in vivo pharmacology and/or efficacy. Different crystalline forms occur where the same composition of matter crystallizes in a different lattice arrangement resulting in different thermodynamic properties and stabilities specific to the particular polymorph form. In cases where two or more crystalline forms can be produced, it is desirable to have a method to make each of the crystalline forms in pure form. In deciding which crystalline form is preferable, the numerous properties of the crystal forms must be compared and the preferred crystal form chosen based on the many physical property variables. It is entirely possible that one crystalline form can be preferable in some circumstances where certain aspects such as ease of preparation, stability, etc. are deemed to be critical. In other situations, a different crystalline form maybe preferred for greater solubility and/or superior pharmacokinetics. Moreover, because of the potential advantages associated with one pure crystalline form, it is desirable to prevent or minimize polymorphic conversion (i.e., conversion of one crystal form to another; or conversion between one crystal form and amorphous form) when two or more crystalline forms of one substance can exist. Such polymorphic conversion can occur during both the preparation of formulations containing the crystalline form, and during storage of a pharmaceutical dosage form containing a crystal form. Because improved drug formulations showing, for example, better bioavailability or better stability are consistently sought, there is an ongoing need for new or purer solid (e.g. crystalline) forms of existing drug molecules. The novel solid (e.g. crystalline) forms of tris salt of Compound 1 described herein are directed toward this and other important ends.

### SUMMARY OF THE INVENTION

The present invention provides a solid form, for example a hydrate (e.g. a monohydrate) crystalline form of 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, tris salt (e.g. Form 2 or Form 3) or an amorphous form, characterized according to the powder X-ray diffraction data, ¹³C solid state NMR data, and/or optionally single crystal spectrum data provided herein.

The present invention further provides compositions containing a hydrate (e.g. a monohydrate) crystalline form of the invention (e.g. Form 2 or Form 3).

The present invention further provides a method for preparing a hydrate (e.g. a monohydrate) crystalline form of the invention (e.g. Form 2 or Form 3), for example, a method for preparing Form 3 comprising slurrying an anhydrous crystalline form (e.g. Form A) of tris salt of Compound 1 in a mixed solvent to form the monohydrate crystalline form, wherein the mixed solvent comprises water and acetonitrile.

The present invention further provides a method for treating a disease or disorder comprising administering to a mammal in need of such treatment a therapeutically effective amount of a hydrate (e.g. a monohydrate) crystalline form of the invention (e.g. Form 2 or Form 3), wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, a cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

The present invention further provides an amorphous form of 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, tris salt, a pharmaceutical composition thereof, and use thereof in treating a disease or disorder modulated by GLP-1R.

### BRIEF DESCRIPTION OF FIGURES

FIG. 1 shows an observed powder X-ray diffraction pattern for an anhydrous crystalline form (Form A) of tris salt of Compound 1 carried out on a Bruker AXS D8 Endeavor diffractometer equipped with a Cu radiation source.

FIG. 2 shows an observed ¹³C ssNMR pattern of Form A of tris salt of Compound 1 conducted on a Bruker-BioSpin CPMAS probe positioned into a Bruker-BioSpin Avance III 500 MHz (¹H frequency) NMR spectrometer. The peaks marked by hashed marks and the gray shaded box are spinning sidebands.

FIG. 3 shows an illustrative single crystal structure of a monohydrate crystalline form (Form 2) of tris salt of Compound 1.

FIG. 4 shows a calculated/simulated PXRD pattern of Form 2 of tris salt of Compound 1 based on the information from its single crystal X-ray data analysis.

FIG. 5 shows an illustrative single crystal structure of a monohydrate crystalline form (Form 3) of tris salt of Compound 1.

FIG. 6 shows an observed powder X-ray diffraction pattern for Form 3 of tris salt of Compound 1 carried out on a Bruker AXS D8 Endeavor diffractometer equipped with a Cu radiation source.

FIG. 7 shows an observed ¹³C ssNMR pattern of Form 3 of tris salt of Compound 1 conducted on a Bruker-BioSpin CPMAS probe positioned into a Bruker-BioSpin Avance III 500 MHz (¹H frequency) NMR spectrometer. The peaks marked by hashed marks and the gray shaded box are spinning sidebands.

### DETAILED DESCRIPTION OF THE INVENTION

In a first aspect, the present invention provides a hydrate (e.g. a monohydrate) cystalline form of tris salt of Compound 1, which can be identified by its unique solid state signatures with respect to, for example, single crystal X-ray data, powder X-ray diffraction pattern (PXRD), and other solid state methods such as solid state NMR. A hydrate crystalline form of tris salt of Compound 1 disclosed herein refers to a crystalline material/complex that includes both tris salt of Compound 1 and water (hydrate water) in the crystal lattice of the crystalline material/complex.

In a second aspect, the present invention provides a monohydrate cystalline form of tris salt of Compound 1, designated as Form 2 herein. The monohydrate cystalline form of tris salt of Compound 1 (Form 2) can be identified by its unique solid state signatures with respect to, for example, single crystal X-ray data, powder X-ray diffraction pattern (PXRD), and other solid state methods.

Form 2 can be prepared by slow solvent evaporation of a solution of tris salt of Compound 1 in a solvent to precipitate Form 2, wherein the solvent is about 3% to about 10% (e.g. about 2% to about 5%, or about 3% to about 4%, v/v) water in a protic organic solvent (that is miscible with water), for example, an alcohol such as methanol or ethanol. In some embodiments, Form 2 is prepared by slow solvent evaporation of a solution of tris salt of Compound 1 in a solvent, wherein the solvent is about 2% to about 5% (e.g. or about 3% to about 4%, v/v) water in methanol. In some embodiments, the solution of the tris salt of Compound 1 is generated in situ, for example, by mixing a solution of Compound 1 in a protic organic solvent that is miscible with water (e.g. an alcohol such as methonal) with an aqueous solution of tris.

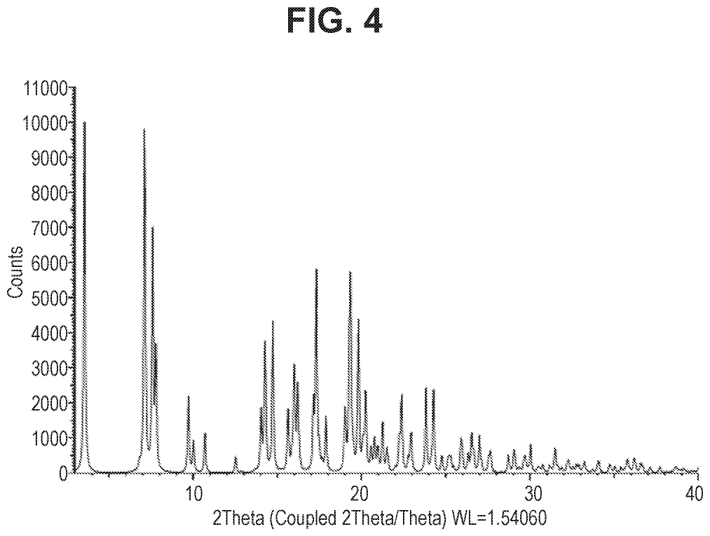

Form 2 has a calculated/simulated PXRD pattern substantially the same as that shown in FIG. 4. Simulated peak locations and intensities for the PXRD pattern in FIG. 4 are provided in Table E2-5. Some characteristic PXRD peaks of Form 2, expressed as 2θ±0.2° 2θ are at 7.1, 7.6, 10.7, and 19.4 (diffraction angles). In some embodiments, Form 2 has a powder X-ray diffraction pattern (PXRD) comprising at least one peak, in terms of 2θ±0.2° 2θ, at 7.1, 7.6, 10.7, and 19.4. In some embodiments, Form 2 has a PXRD comprising at least two peaks, in terms of 2θ±0.2° 2θ, at 7.1, 7.6, 10.7, and 19.4. In some embodiments, Form 2 has a PXRD comprising at least two peaks, in terms of 2θ±0.2° 2θ, at 7.1 and 10.7. In some embodiments, Form 2 has a PXRD comprising at least two peaks, in terms of 2θ±0.2° 2θ, at 7.1 and 7.6. In some embodiments, Form 2 has a PXRD comprising at least three peaks, in terms of 2θ±0.2° 2θ, at 7.1, 7.6, and 10.7. In some embodiments, Form 2 has a PXRD comprising at least three peaks, in terms of 2θ±0.2° 2θ, at 7.1, 7.6, and 19.4. In some embodiments, Form 2 has a PXRD comprising at least four peaks, in terms of 2θ±0.2° 2θ, at 7.1, 7.6, 10.7, and 19.4.

In a third aspect, the present invention provides a monohydrate cystalline form of tris salt of Compound 1, designated as Form 3 herein. The monohydrate cystalline form of tris salt of Compound 1 (Form 3) can be identified by its unique solid state signatures with respect to, for example, single crystal X-ray data, PXRD, ¹³C ssNMR, and other solid state methods.

Form 3 can be prepared by slurry to slurry conversion. A slurry of Form A (an anhydrous form of tris salt of Compound 1) in a solvent system is stirred for a period sufficiently long to convert Form A to Form 3, wherein the solvent system includes an aprotic organic solvent (e.g. acetonitrile or tetrahydrofuran) and water. In some embodiments, the solvent system includes acetonitrile and water, and the ratio of water to acetonitrile in the solvent system is from about 2:98 to about 15:85 (e.g. about 8:92 v/v). In some embodiments, the ratio of the solvent system (in mL) to Form A (in gram) is about 10:1 to about 40:1, for example, about 15:1 to about 30:1, or about 25:1 to about 35:1. The slurry to slurry conversion can be carried out at room temperature with sufficient mixing/stirring. Preparation of the starting material Form A (and its physical characteristic property) is shown in Example 1. The conversion of Form A to Form 3 can be monitored/assessed by PXRD.

Alternatively, Form 3 can be prepared by vapor diffusion of acetonitrile into a concentrated (e.g. saturated) solution of tris salt of Compound 1 in a solvent system, wherein the solvent system is a mixture of acetonitrile and water, and the percentage of water in the solvent system is more than about 10% by volume, for example about 15%. In some embodiment, the tris salt of Compound 1 in the saturated solution can the concentrated (e.g. saturated) solution can be generated in situ, for example, by mixing a solution of Compound 1 in acetonitrile with an aqueous solution of tris (for example, about 1:1 molar ratio). Alternatively, acetonitrile can be substituted by another aprotic organic solvent that is miscible with water (e.g. tetrahydrofuran) in the vapor diffusion method described herein [i.e., Form 3 can be prepared by vapor diffusion of an aprotic solvent into a concentrated (e.g. saturated) solution of tris salt of Compound 1 in a solvent system, wherein the solvent system is a mixture of the aprotic organic solvent and water].

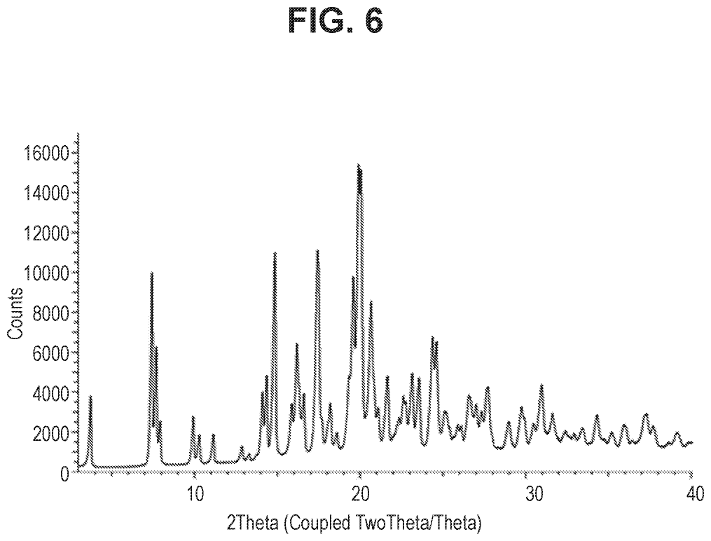

Form 3 has a PXRD pattern substantially the same as that shown in FIG. 6. Peak locations and intensities for the PXRD pattern in FIG. 6 are provided in Table E3-5. Some characteristic PXRD peaks of Form 3, expressed as 2θ±0.2° 2θ are at 3.7, 7.4, 9.9, 14.8, and 20.6 (diffraction angles). In some embodiments, Form 3 has a PXRD comprising at least one, two, three, or four peaks, in terms of 2θ±0.2° 2θ, at 3.7, 7.4, 9.9, 14.8, and 20.6. In some embodiments, Form 3 has a PXRD comprising at least two or three peaks, in terms of 2θ±0.2° 2θ, at 3.7, 7.4, 9.9, 14.8, and 20.6. In some embodiments, Form 3 has a PXRD comprising two peaks, in terms of 2θ±0.2° 2θ, at 7.4 and 14.8. In some embodiments, Form 3 has a PXRD comprising three peaks, in terms of 2θ±0.2° 2θ, at 3.7, 7.4, and 14.8. In some embodiments, Form 3 has a PXRD comprising four peaks, in terms of 2θ±0.2° 2θ, at 3.7, 7.4, 14.8, and 20.6. In some embodiments, Form 3 has a PXRD comprising five peaks, in terms of 2θ±0.2° 2θ, at 3.7, 7.4, 9.9, 14.8, and 20.6. In some embodiments, Form 3 has a PXRD comprising peaks, in terms of 2θ±0.2° 2θ, at 3.7, 7.4, 9.9, 11.1, 14.8, 18.2, 20.6, 23.5, 24.3, and 24.6.

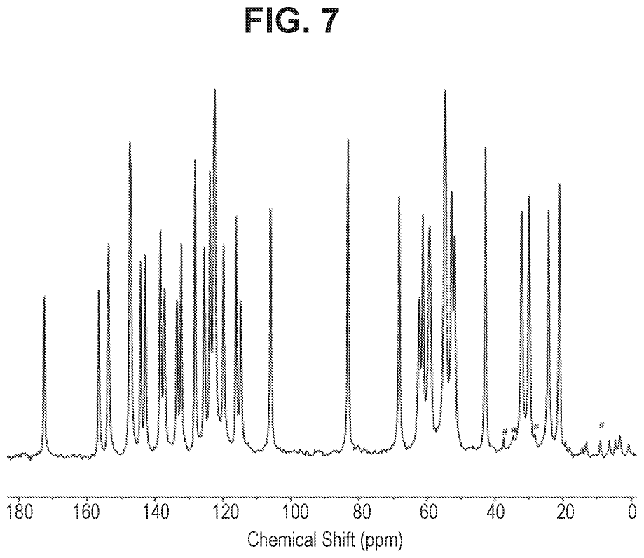

Form 3 has a ¹³C ssNMR spectrum substantially the same as that shown in FIG. 7. ¹³C Chemical shifts (±0.2 ppm) of Form 3 as shown in FIG. 7 are listed in Table E3-6. Some characteristic ¹³C ssNMR chemical shifts of Form 3, expressed as ppm, are at 42.8, 54.7, 128.2, 138.4 and 156.6±0.2 ppm.

In some embodiments, Form 3 has a ¹³C ssNMR spectrum comprising chemical shifts at 54.7 and 138.4±0.2 ppm. In some embodiments, Form 3 has a ¹³C ssNMR spectrum comprising chemical shifts at 54.7, 138.4 and 156.6 ppm±0.2 ppm.

In a fourth aspect, the present invention further provides an amorphous form of tris salt of Compound 1. The amorphous form of tris salt of Compound 1 does not give distinctive powder X-ray diffraction patterns (i.e., it PXRD does not have sharp peaks as in a PXRD for Form A or Form 3). The amorphous form of tris salt of Compound 1 can be prepared, for example, by a lyophilization process (starting from a solution of tris salt of Compound 1).

Any solid form of the present invention can be substantially pure. As used herein, the term “substantially pure” with reference to a particular solid form (e.g. a crystalline form) means that the particular solid form (e.g. the crystalline form) includes less than 15%, less than 10%, less than 5%, less than 3%, or less than 1% by weight of any other physical form of tris salt of Compound 1.

The term “substantially the same” when used to describe X-ray powder diffraction patterns is meant to include patterns in which peaks are within a standard deviation of +/−0.2° 2θ.

The term “substantially the same” when used to describe ¹³C ssNMR spectrum meant to include ¹³C ssNMR spectra in which chemical shifts are within a standard deviation of +/−0.2 ppm.

The term “about” generally means within 10%, preferably within 5%, and more preferably within 1% of a given value or range. Alternatively, the term “about” means within an acceptable standard error of the mean, when considered by one skilled in the art.

The term “tris” means 1,3-dihydroxy-2-(hydroxymethyl)propan-2-amine, also known as THAM, tromethamine, or 2-amino-2-(hydroxymethyl)propane-1,3-diol.

Tris salt of Compound 1 means a salt of Compound 1 made using 1,3-dihydroxy-2-(hydroxymethyl)propan-2-amine. The tris is associated with the carboxylic acid moiety of Compound 1. Unless otherwise stated, when referencing the tris salt of Compound 1, the counterion and Compound 1 are in a stoichiometric ratio of about 1:1 (i.e. from 0.9:1.0 to 1.0:0.9, for example, from 0.95:1.00 to 1.00:0.95). Another chemical name for tris salt of Compound 1 is 1,3-dihydroxy-2-(hydroxymethyl)propan-2-aminium 2-((4-((S)-2-(5-Chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylate, which can also be represented, for example, by one of the following structures.

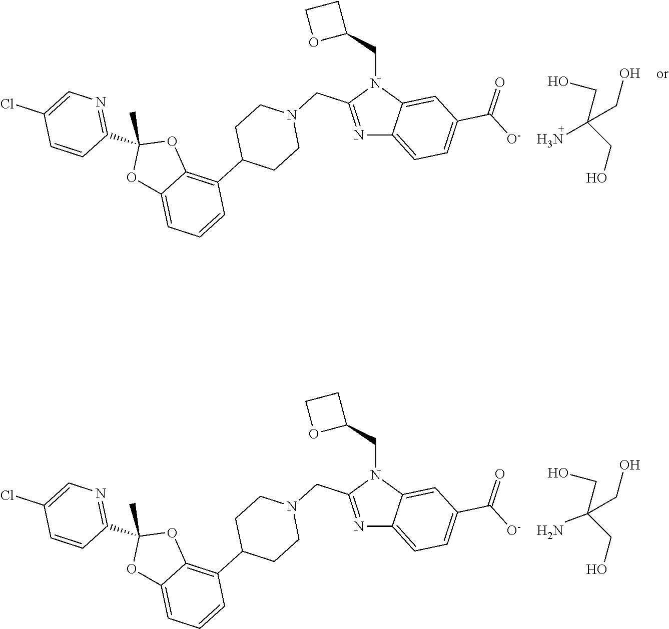 [SMILES: C[C@]1(c2ccc(Cl)cn2)Oc2cccc(C3CCN(Cc4nc5ccc(C(=O)[O-])cc5n4C[C@@H]4CCO4)CC3)c2O1 ; NC(CO)(CO)CO ; C[C@]1(c2ccc(Cl)cn2)Oc2cccc(C3CCN(Cc4nc5ccc(C(=O)[O-])cc5n4C[C@@H]4CCO4)CC3)c2O1 ; NC(CO)(CO)CO]

Those skilled in the art would readily understand that multiple nomenclatures can be used to name a same compound (including a same salt).

The term “monohydrate” when used to describe a crystalline form of a compound (or a salt) means that the stoichiometric ratio of the hydrate water to the compound (or salt) is about 1:1 (for example, from 0.9:1.0 to 1.1:1.0).

In another embodiment, the invention provides a pharmaceutical composition comprising a crystalline form of the invention (e.g. Form 3), in admixture with at least one pharmaceutically acceptable excipient. This would include a pharmaceutical composition comprising a crystalline form of the invention (e.g. Form 3), as defined in any of the embodiments described herein, in admixture with at least one pharmaceutically acceptable excipient and one or more other therapeutic agent discussed herein.

In another embodiment, the invention provides a pharmaceutical composition comprising an amorphous form of the invention, in admixture with at least one pharmaceutically acceptable excipient. This would include a pharmaceutical composition comprising an amorphous form of the invention, in admixture with at least one pharmaceutically acceptable excipient and one or more other therapeutic agent discussed herein.

In another embodiment, the invention provides a pharmaceutical composition comprising a therapeutically effective amount of tris salt of Compound 1 and a pharmaceutically acceptable carrier, wherein at least 5%, 10%, 15%, 20%, 30%, 40%, 50%, 60%, 70%, 80%, 85%, 90%, 95%, 98%, or 99% of the tris salt of Compound 1 is present as one of solid forms of the invention (e.g., Form 2, Form 3, or amorphous form).

In another embodiment, the invention provides a pharmaceutical composition comprising a therapeutically effective amount of tris salt of Compound 1 and a pharmaceutically acceptable carrier, wherein the tris salt of Compound 1 is present as at least two solid forms, for example, a crystalline form and an amorphous form.

In another embodiment, the invention provides a pharmaceutical composition comprising a therapeutically effective amount of tris salt of Compound 1 and a pharmaceutically acceptable carrier, wherein the tris salt of Compound 1 is present as at least two solid forms, for example, a crystalline form of the invention (e.g., Form 2 or Form 3) and an amorphous form.

In a further embodiment, the invention provides a pharmaceutical composition comprising a therapeutically effective amount of tris salt of Compound 1 and a pharmaceutically acceptable carrier, wherein the tris salt of Compound 1 is present in two solid forms, one of which is an amorphous form and other is Form 3.

In another embodiment, the invention provides a pharmaceutical composition comprising a therapeutically effective amount of tris salt of Compound 1 and a pharmaceutically acceptable carrier, wherein the tris salt of Compound 1 is present in two solid forms, one of which is amorphous and other is Form 2.

In another embodiment, the invention provides a pharmaceutical composition comprising a therapeutically effective amount of tris salt of Compound 1 and a pharmaceutically acceptable carrier, wherein the tris salt of Compound 1 is present in two solid forms, one of which is Form A and other is Form 3.

The invention also includes the following embodiments:

a solid form of the invention (e.g. Form 2, Form 3, or amorphous form), as defined in any of the embodiments described herein, for use as a medicament;

a solid form of the invention (e.g. Form 2, Form 3, or amorphous form), as defined in any of the embodiments described herein, for use in the prevention and/or treatment of cardiometabolic and associated diseases discussed herein, including T2DM, pre-diabetes, obesity, NASH (e.g. NASH with fibrosis), NAFLD, and cardiovascular disease;

a method of treating a disease for which an agonist of GLP-1R is indicated, in a subject in need of such prevention and/or treatment, comprising administering to the subject a therapeutically effective amount of a solid form of the invention (e.g. Form 2, Form 3, or amorphous form), as defined in any of the embodiments described herein;

the use of a solid form of the invention (e.g. Form 2, Form 3, or amorphous form), as defined in any of the embodiments described herein, for the manufacture of a medicament for treating a disease or condition for which an agonist of the GLP-1R is indicated;

a solid form of the invention (e.g. Form 2, Form 3, or amorphous form), as defined in any of the embodiments described herein, for use in the treatment of a disease or condition for which an agonist of GLP-1R is indicated; or

a pharmaceutical composition for the treatment of a disease or condition for which an agonist of the GLP-1R is indicated, comprising a solid form of the invention (e.g. Form 2, Form 3, or amorphous form), as defined in any of the embodiments described herein.

The invention also includes the following embodiments:

a crystalline form of the invention (e.g. Form 3), as defined in any of the embodiments described herein, for use as a medicament;

a crystalline form of the invention (e.g. Form 3), as defined in any of the embodiments described herein, for use in the prevention and/or treatment of cardiometabolic and associated diseases discussed herein, including T2DM, pre-diabetes, obesity, NASH (e.g. NASH with fibrosis), NAFLD, and cardiovascular disease;

a method of treating a disease for which an agonist of GLP-1R is indicated, in a subject in need of such prevention and/or treatment, comprising administering to the subject a therapeutically effective amount of a crystalline form of the invention (e.g. Form 3), as defined in any of the embodiments described herein;

the use of a crystalline form of the invention (e.g. Form 3), as defined in any of the embodiments described herein, for the manufacture of a medicament for treating a disease or condition for which an agonist of the GLP-1R is indicated;

a crystalline form of the invention (e.g. Form 3), as defined in any of the embodiments described herein, for use in the treatment of a disease or condition for which an agonist of GLP-1R is indicated; or

a pharmaceutical composition for the treatment of a disease or condition for which an agonist of the GLP-1R is indicated, comprising a crystalline form of the invention (e.g. Form 3), as defined in any of the embodiments described herein.

Every example of solid forms of the invention may be claimed individually or grouped together in any combination with any number of each and every embodiment described herein.

The invention also relates to a pharmaceutical composition comprising a solid form of the invention (e.g. Form 2, Form 3, or amorphous form), as defined in any of the embodiments described herein, for use in the treatment and/or prevention of cardiometabolic and associated diseases discussed herein, including T2DM, pre-diabetes, obesity, NASH (e.g. NASH with fibrosis), NAFLD, and cardiovascular disease.

The invention also relates to a pharmaceutical composition comprising a crystalline form of the invention (e.g. Form 3), as defined in any of the embodiments described herein, for use in the treatment and/or prevention of cardiometabolic and associated diseases discussed herein, including T2DM, pre-diabetes, obesity, NASH (e.g. NASH with fibrosis), NAFLD, and cardiovascular disease.

Another embodiment of the invention concerns a solid form of the invention (e.g. Form 2, Form 3, or amorphous form), for example a crystalline form of the invention (e.g. Form 3), as defined in any of the embodiments described herein, for use in the treatment and/or prevention of diseases and/or disorders for which a GLP-1R agonist is indicated including, diabetes (T1D and/or T2DM, including pre-diabetes), idiopathic T1D (Type 1 b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease (e.g., acute kidney disorder, tubular dysfunction, proinflammatory changes to the proximal tubules), diabetic retinopathy, adipocyte dysfunction, visceral adipose deposition, sleep apnea, obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain from use of other agents (e.g., from use of steroids and antipsychotics), excessive sugar craving, dyslipidemia (including hyperlipidemia, hypertriglyceridemia, increased total cholesterol, high LDL cholesterol, and low HDL cholesterol), hyperinsulinemia, NAFLD (including related diseases such as steatosis, NASH, fibrosis, NASH with fibrosis, cirrhosis, and hepatocellular carcinoma), cardiovascular disease, atherosclerosis (including coronary artery disease), peripheral vascular disease, hypertension, endothelial dysfunction, impaired vascular compliance, congestive heart failure, myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction (e.g., alcohol and/or drug abuse).

Room temperature: RT (15 to 25° C.).

Methanol: MeOH.

Ethanol: EtOH.

Isopropanol: iPrOH.

Ethyl acetate: EtOAc.

Tetrahydrofuran: THF.

Toluene: PhCH₃.

Cesium carbonate: Cs₂CO₃.

Lithium bis(trimethylsilyl)amide: LiHMDS.

Sodium t-butoxide: NaOtBu.

Potassium t-butoxide: KOtBu.

Lithium diisopropylamide: LDA.

Triethylamine: NEt₃.

N,N-diisopropylethyl amine: DIPEA.

Potassium carbonate: K₂CO₃.

Dimethyl formamide: DMF.

Dimethyl acetamide: DMAc.

Dimethyl sulfoxide: DMSO.

N-Methyl-2-pyrrolidinone: NMP.

Sodium hydride: NaH.

Trifluoroacetic acid: TFA.

Trifluoroacetic anhydride: TFAA.

Acetic anhydride: Ac₂O.

Dichloromethane: DCM.

1,2-Dichloroethane: DCE.

Hydrochloric acid: HCl.

1,8-Diazabicyclo[5.4.0]undec-7-ene: DBU.

Borane-dimethylsulfide complex: BH₃-DMS.

Borane-tetrahydrofuran complex: BH₃-THF.

Lithium aluminum hydride: LAH.

Acetic acid: AcOH.

Acetonitrile: MeCN.

p-Toluenesulfonic acid: pTSA.

Dibenzylidine acetone: DBA.

2,2′-Bis(diphenylphosphino)-1,1′-binaphthalene: BINAP.

1,1′-Ferrocenediyl-bis(diphenylphosphine): dppf.

1,3-Bis(diphenylphosphino)propane: DPPP.

3-Chloroperbenzoic acid: m-CPBA.

Tert-Butyl methyl ether: MTBE.

Methanesulfonyl: Ms.

N-Methylpyrrolidinone: NMP.

Thin layer chromatography: TLC.

Supercritical fluid chromatography: SFC.

4-(Dimethylamino)pyridine: DMAP.

Tert-Butyloxycarbonyl: Boc.

Triphenylphospine: Ph₃P.

1-[Bis(dimethylamino)methylene]-1H-1,2,3-triazolo[4,5-b]pyridinium 3-oxid hexafluorophosphate: HATU.

Petroleum ether: PE.

2-(1H-Benzotriazole-1-yl)-1,1,3,3-tetramethyluronium hexafluorophosphate: HBTU.

2-Amino-2-(hydroxymethyl)propane-1,3-diol: tris.

tris(dibenzylideneacetone)dipalladium: Pd₂(dba)₃

¹H Nuclear magnetic resonance (NMR) spectra were in all cases consistent with the proposed structures. Characteristic chemical shifts (δ) are given in parts-per-million relative to the residual proton signal in the deuterated solvent (CHCl₃ at 7.27 ppm; CD₂HOD at 3.31 ppm; MeCN at 1.94 ppm; DMSO at 2.50 ppm) and are reported using conventional abbreviations for designation of major peaks: e.g. s, singlet; d, doublet; t, triplet; q, quartet; m, multiplet; br, broad. The symbol \hat{ } denotes that the ¹H NMR peak area was assumed because the peak was partially obscured by water peak. The symbol \hat{ }\hat{ } denotes that the ¹H NMR peak area was assumed because the peak was partially obscured by solvent peak.

The compounds and intermediates described below were named using the naming convention provided with ACD/ChemSketch 2012, ChemDraw, File Version C10H41, Build 69045 (Advanced Chemistry Development, Inc., Toronto, Ontario, Canada). The naming convention provided with ACD/ChemSketch 2012 is well known by those skilled in the art and it is believed that the naming convention provided with ACD/ChemSketch 2012 generally comports with the IUPAC (International Union for Pure and Applied Chemistry) recommendations on Nomenclature of Organic Chemistry and the CAS Index rules. One will note that the chemical names may have only parentheses or may have parentheses and brackets. The stereochemical descriptors may also be placed at different locations within the name itself, depending on the naming convention. One of ordinary skill in the art will recognize these formatting variations and understand they provide the same chemical structure.

Pharmaceutically acceptable salts include acid addition and base salts.

Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts.

Suitable base salts are formed from bases which form non-toxic salts. Examples include the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-Amino-2-(hydroxymethyl)propane-1,3-diol (tris or tromethamine) and zinc salts.

Hemisalts of acids and bases may also be formed, for example, hemisulfate and hemicalcium salts. For a review on suitable salts, see Handbook of Pharmaceutical Salts: Properties, Selection, and Use by Stahl and Wermuth (Wiley-VCH, 2002).

Pharmaceutically acceptable salts may be prepared by one or more of three methods:
(i) by reacting a compound with the desired acid or base;
(ii) by removing an acid- or base-labile protecting group from a suitable precursor of a compound or by ring-opening a suitable cyclic precursor, for example, a lactone or lactam, using the desired acid or base; or
(iii) by converting one salt of a compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

Compounds and pharmaceutically acceptable salts, may exist in unsolvated and solvated forms. The term ‘solvate’ is used herein to describe a molecular complex comprising a compound or its salt, and one or more pharmaceutically acceptable solvent molecules, for example, ethanol. The term ‘hydrate’ is employed when said solvent is water. For example, a hydrate crystalline form of tris salt of Compound 1 disclosed herein refers to a crystalline material/complex that includes both tris salt of Compound 1 and water (hydrate water) in the crystal lattice of the crystalline material/complex.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

A compound may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Some compounds may exhibit polymorphism and/or one or more kinds of isomerism (e.g. optical, geometric or tautomeric isomerism). The crystalline forms of the inventions may also be isotopically labelled. Such variation is implicit to Compound 1 or its salt defined as they are by reference to their structural features and therefore within the scope of the invention.

Compounds containing one or more asymmetric carbon atoms can exist as two or more stereoisomers. Where a compound contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds containing, for example, an imino, keto, or oxime group, or so-called valence tautomerism in compounds which contain an aromatic moiety. It follows that a single compound may exhibit more than one type of isomerism.

Certain pharmaceutically acceptable salts of Compound 1 may also contain a counterion which is optically active (e.g. d-lactate or l-lysine) or racemic (e.g. dl-tartrate or dl-arginine).

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, a racemic precursor containing a chiral ester may be separated by enzymatic resolution (see, for example, Int J Mol Sci 29682-29716 by A. C. L. M. Carvaho et. al. (2015)). In the case where a compound contains an acidic or basic moiety, a salt may be formed with an optically pure base or acid such as 1-phenylethylamine or tartaric acid. The resulting diastereomeric mixture may be separated by fractional crystallization and one or both of the diastereomeric salts converted to the corresponding pure enantiomer(s) by means well known to a skilled person. Alternatively, the racemate (or a racemic precursor) may be covalently reacted with a suitable optically active compound, for example, an alcohol, amine or benzylic chloride. The resulting diastereomeric mixture may be separated by chromatography and/or fractional crystallization by means well known to a skilled person to give the separated diastereomers as single enantiomers with 2 or more chiral centers. Chiral compounds (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (SFC with Packed Columns), pp. 223-249 and references cited therein). In some relevant examples herein, columns were obtained from Chiral Technologies, Inc, West Chester, Pa., USA, a subsidiary of Daicel® Chemical Industries, Ltd., Tokyo, Japan.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

It must be emphasised that Compound 1 and its salts have been drawn herein in a single tautomeric form, all possible tautomeric forms are included within the scope of the invention.

The present invention includes all pharmaceutically acceptable isotopically-labeled Compound 1 or a salt thereof wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, nitrogen, such as ¹³N and ¹⁵N, and oxygen, such as ¹⁵O, ¹⁷O and ¹⁸O.

Certain isotopically-labelled Compound 1 or a salt thereof, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

### Administration and Dosing

Typically, a compound (in a crystalline form) of the invention is administered in an amount effective to treat a condition as described herein. The compounds of the invention can be administered as compound per se, or alternatively, as a pharmaceutically acceptable salt.

For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, or topically.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention and/or compositions containing said compounds is based on a variety of factors, including the type, age, weight, sex and medical condition of the patient; the severity of the condition; the route of administration; and the activity of the particular compound employed. Thus the dosage regimen may vary widely. In one embodiment, the total daily dose of a compound of the invention is typically from about 0.001 to about 100 mg/kg (i.e., mg compound of the invention per kg body weight) for the treatment of the indicated conditions discussed herein. In another embodiment, total daily dose of the compound of the invention is from about 0.01 to about 30 mg/kg, and in another embodiment, from about 0.03 to about 10 mg/kg, and in yet another embodiment, from about 0.1 to about 3. It is not uncommon that the administration of the compounds of the invention will be repeated a plurality of times in a day (typically no greater than 4 times). Multiple doses per day typically may be used to increase the total daily dose, if desired.

For oral administration, the compositions may be provided in the form of tablets containing 0.1, 0.5, 1.0, 2.5, 5.0, 10.0, 15.0, 25.0, 30.0 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Suitable subjects according to the invention include mammalian subjects. In one embodiment, humans are suitable subjects. Human subjects may be of either gender and at any stage of development.

Pharmaceutical Compositions

In another embodiment, the invention comprises pharmaceutical compositions. Such pharmaceutical compositions comprise a compound of the invention presented with a pharmaceutically acceptable carrier. Other pharmacologically active substances can also be present. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Pharmaceutically acceptable substances such as wetting agents or minor amounts of auxiliary substances such as wetting or emulsifying agents, preservatives or buffers, which enhance the shelf life or effectiveness of the antibody or antibody portion.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g. intravenous, subcutaneous, intraperitoneal, intramuscular). In another embodiment, the antibody is administered by intravenous infusion or injection. In yet another embodiment, the antibody is administered by intramuscular or subcutaneous injection.

Oral administration of a solid dose form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dose form is sub-lingual, such as, for example, a lozenge. In such solid dosage forms, the compounds of the invention are ordinarily combined with one or more adjuvants. Such capsules or tablets may contain a controlled release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the invention comprises a parenteral dose form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Co-Administration

The compounds of the invention can be used alone, or in combination with other therapeutic agents. The invention provides any of the uses, methods or compositions as defined herein wherein the compound of any embodiment herein, or pharmaceutically acceptable salt thereof, or pharmaceutically acceptable solvate of said compound or salt, is used in combination with one or more other therapeutic agent discussed herein. This would include a pharmaceutical composition for the treatment of a disease or condition for which an agonist of the GLP-1R is indicated, comprising a crystalline form of the invention, as defined in any of the embodiments described herein, and one or more other therapeutic agent discussed herein.

The administration of two or more compounds “in combination” means that all of the compounds are administered closely enough in time that each may generate a biological effect in the same time frame. The presence of one agent may alter the biological effects of the other compound(s). The two or more compounds may be administered simultaneously, concurrently or sequentially. Additionally, simultaneous administration may be carried out by mixing the compounds prior to administration or by administering the compounds at the same point in time but as separate dosage forms at the same or different site of administration.

The phrases “concurrent administration,” “co-administration,” “simultaneous administration,” and “administered simultaneously” mean that the compounds are administered in combination.

In another embodiment, the invention provides methods of treatment that include administering compounds of the present invention in combination with one or more other pharmaceutical agents, wherein the one or more other pharmaceutical agents may be selected from the agents discussed herein.

In one embodiment, the compounds of this invention are administered with an antidiabetic agent including but not limited to a biguanide (e.g., metformin), a sulfonylurea (e.g., tolbutamide, glibenclamide, gliclazide, chlorpropamide, tolazamide, acetohexamide, glyclopyramide, glimepiride, or glipizide), a thiazolidinedione (e.g., pioglitazone, rosiglitazone, or lobeglitazone), a glitazar (e.g., saroglitazar, aleglitazar, muraglitazar or tesaglitazar), a meglitinide (e.g., nateglinide, repaglinide), a dipeptidyl peptidase 4 (DPP-4) inhibitor (e.g., sitagliptin, vildagliptin, saxagliptin, linagliptin, gemigliptin, anagliptin, teneligliptin, alogliptin, trelagliptin, dutogliptin, or omarigliptin), a glitazone (e.g., pioglitazone, rosiglitazone, balaglitazone, rivoglitazone, or lobeglitazone), a sodium-glucose linked transporter 2 (SGLT2) inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a GPR40 agonist (FFAR1/FFA1 agonist, e.g. fasiglifam), glucose-dependent insulinotropic peptide (GIP) and analogues thereof, an alpha glucosidase inhibitor (e.g. voglibose, acarbose, or miglitol), or an insulin or an insulin analogue, including the pharmaceutically acceptable salts of the specifically named agents and the pharmaceutically acceptable solvates of said agents and salts.

In another embodiment, the compounds of this invention are administered with an anti-obesity agent including but not limited to peptide YY or an analogue thereof, a neuropeptide Y receptor type 2 (NPYR2) agonist, a NPYR1 or NPYR5 antagonist, a cannabinoid receptor type 1 (CB1R) antagonist, a lipase inhibitor (e.g., orlistat), a human proislet peptide (HIP), a melanocortin receptor 4 agonist (e.g., setmelanotide), a melanin concentrating hormone receptor 1 antagonist, a farnesoid X receptor (FXR) agonist (e.g. obeticholic acid), zonisamide, phentermine (alone or in combination with topiramate), a norepinephrine/dopamine reuptake inhibitor (e.g., buproprion), an opioid receptor antagonist (e.g., naltrexone), a combination of norepinephrine/dopamine reuptake inhibitor and opioid receptor antagonist (e.g., a combination of bupropion and naltrexone), a GDF-15 analog, sibutramine, a cholecystokinin agonist, amylin and analogues therof (e.g., pramlintide), leptin and analogues thereof (e.g., metroleptin), a serotonergic agent (e.g., lorcaserin), a methionine aminopeptidase 2 (MetAP2) inhibitor (e.g., beloranib or ZGN-1061), phendimetrazine, diethylpropion, benzphetamine, an SGLT2 inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a dual SGLT2/SGLT1 inhibitor, a fibroblast growth factor receptor (FGFR) modulator, an AMP-activated protein kinase (AMPK) activator, biotin, a MAS receptor modulator, or a glucagon receptor agonist (alone or in combination with another GLP-1R agonist, e.g., liraglutide, exenatide, dulaglutide, albiglutide, lixisenatide, or semaglutide), including the pharmaceutically acceptable salts of the specifically named agents and the pharmaceutically acceptable solvates of said agents and salts.

In another embodiment, the compounds of this invention are administered in combination with one or more of the following: an agent to treat NASH including but not limited to PF-05221304, an FXR agonist (e.g., obeticholic acid), a PPAR a/8 agonist (e.g., elafibranor), a synthetic fatty acid-bile acid conjugate (e.g., aramchol), a caspase inhibitor (e.g., emricasan), an anti-lysyl oxidase homologue 2 (LOXL2) monoclonal antibody (e.g., simtuzumab), a galectin 3 inhibitor (e.g., GR-MD-02), a MAPK5 inhibitor (e.g., GS-4997), a dual antagonist of chemokine receptor 2 (CCR2) and CCR5 (e.g., cenicriviroc), a fibroblast growth factor 21 (FGF21) agonist (e.g., BMS-986036), a leukotriene D4 (LTD4) receptor antagonist (e.g., tipelukast), a niacin analogue (e.g., ARI 3037MO), an ASBT inhibitor (e.g., volixibat), an acetyl-CoA carboxylase (ACC) inhibitor (e.g., NDI 010976 or PF-05221304), a ketohexokinase (KHK) inhibitor, a diacylglyceryl acyltransferase 2 (DGAT2) inhibitor, a CB1 receptor antagonist, an anti-CB1R antibody, or an apoptosis signal-regulating kinase 1 (ASK1) inhibitor, including the pharmaceutically acceptable salts of the specifically named agents and the pharmaceutically acceptable solvates of said agents and salts.

Some specific compounds that can be used in combination with the compounds of the present invention for treating diseases or disorders described herein (e.g. NASH) include:

4-(4-(1-Isopropyl-7-oxo-1,4,6,7-tetrahydrospiro[indazole-5,4′-piperidine]-1′-carbonyl)-6-methoxypyridin-2-yl)benzoic acid, which is an example of a selective ACC inhibitor and was prepared as the free acid in Example 9 of U.S. Pat. No. 8,859,577, which is the U.S. national phase of International Application No. PCT/IB2011/054119, the disclosures of which are hereby incorporated herein by reference in their entireties for all purposes. Crystal forms of 4-(4-(1-Isopropyl-7-oxo-1,4,6,7-tetrahydrospiro[indazole-5,4′-piperidine]-1′-carbonyl)-6-methoxypyridin-2-yl)benzoic acid, including an anhydrous mono-tris form (Form 1) and a trihydrate of the mono-tris salt (Form 2), are described in International PCT Application No. PCT/IB2018/058966, the disclosure of which is hereby incorporated herein by reference in its entirety for all purposes;

(S)-2-(5-((3-Ethoxypyridin-2-yl)oxy)pyridin-3-yl)-N-(tetrahydrofuran-3-yl)pyrimidine-5-carboxamide, or a pharmaceutically acceptable salt thereof, and its crystalline solid forms (Form 1 and Form 2) is an example of a DGAT2 inhibitor described in Example 1 of U.S. Pat. No. 10,071,992, the disclosure of which is hereby incorporated herein by reference in its entirety for all purposes;

[(1R,5S,6R)-3-{2-[(2S)-2-methylazetidin-1-yl]-6-(trifluoromethyl)pyrimidin-4-yl}-3-azabicyclo[3.1.0]hex-6-yl]acetic acid, or a pharmaceutically acceptable salt thereof, (including a crystalline free acid form thereof) is an example of a ketohexokinase inhibitor and is described in Example 4 of U.S. Pat. No. 9,809,579, the disclosure of which is hereby incorporated herein by reference in its entirety for all purposes; and

the FXR agonist Tropifexor or a pharmaceutically acceptable salt thereof is described in Example 1-1B of U.S. Pat. No. 9,150,568, the disclosure of which is hereby incorporated herein by reference in its entirety for all purposes.

These agents and compounds of the invention can be combined with pharmaceutically acceptable vehicles such as saline, Ringer's solution, dextrose solution, and the like. The particular dosage regimen, i.e., dose, timing and repetition, will depend on the particular individual and that individual's medical history.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or Igs; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Liposomes containing these agents and/or compounds of the invention are prepared by methods known in the art, such as described in U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

These agents and/or the compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing the compound of Formulas I, II, III, IV, or V, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as those used in LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a compound of the invention with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Kits

Another aspect of the invention provides kits comprising a solid form of the invention (e.g. a crystalline form such as Form 3) or pharmaceutical compositions comprising a solid form of the invention (e.g. a crystalline form such as Form 3). A kit may include, in addition to a solid form of the invention (e.g. a crystalline form such as Form 3) or pharmaceutical composition thereof, diagnostic or therapeutic agents. A kit may also include instructions for use in a diagnostic or therapeutic method. In some embodiments, the kit includes a crystalline form of the invention and a diagnostic agent. In other embodiments, the kit includes a crystalline form of the invention, or a pharmaceutical composition thereof.

In yet another embodiment, the invention comprises kits that are suitable for use in performing the methods of treatment described herein. In one embodiment, the kit contains a first dosage form comprising one or more of solid forms of the invention (e.g. a crystalline form such as Form 3) in quantities sufficient to carry out the methods of the invention. In another embodiment, the kit comprises one or more solid forms of the invention (e.g. a crystalline form such as Form 3) in quantities sufficient to carry out the methods of the invention and a container for the dosage and a container for the dosage.

### Preparation

Compound 1, tris salt thereof, and crystalline forms of tris salt of Compound 1, may be prepared by the general and specific methods described below, using the common general knowledge of one skilled in the art of synthetic organic chemistry. Such common general knowledge can be found in standard reference books such as Comprehensive Organic Chemistry, Ed. Barton and Ollis, Elsevier; Comprehensive Organic Transformations: A Guide to Functional Group Preparations, Larock, John Wiley and Sons; and Compendium of Organic Synthetic Methods, Vol. I-XII (published by Wiley-Interscience). The starting materials used herein are commercially available or may be prepared by routine methods known in the art.

In the preparation of the compounds, salts, and crystalline forms of the invention, it is noted that some of the preparation methods described herein may require protection of remote functionality (e.g., primary amine, secondary amine, carboxyl in precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and can typically be removed without chemically altering other functionality in the compounds.

The Schemes described below are intended to provide a general description of the methodology employed in the preparation of the compounds of the present invention. Some of the compounds of the present invention may contain single or multiple chiral centers with the stereochemical designation (R) or (S). It will be apparent to one skilled in the art that all of the synthetic transformations can be conducted in a similar manner whether the materials are enantioenriched or racemic. Moreover the resolution to the desired optically active material may take place at any desired point in the sequence using well known methods such as described herein and in the chemistry literature. For example, intermediates and finals may be separated using chiral chromatographic methods. Alternatively, chiral salts may be utilized to isolate enantiomerically enriched intermediates and final compounds.

### EXAMPLES

The following illustrate the synthesis of non-limiting compounds (including solid forms thereof) of the present invention.

Experiments were generally carried out under inert atmosphere (nitrogen or argon), particularly in cases where oxygen- or moisture-sensitive reagents or intermediates were employed. Commercial solvents and reagents were generally used without further purification. Anhydrous solvents were employed where appropriate, generally AcroSeal® products from Acros Organics, Aldrich® Sure/Seal™ from Sigma-Aldrich, or DriSolv® products from EMD Chemicals. In other cases, commercial solvents were passed through columns packed with 4 Å molecular sieves, until the following QC standards for water were attained: a) <100 ppm for dichloromethane, toluene, N,N-dimethylformamide, and tetrahydrofuran; b) <180 ppm for methanol, ethanol, 1,4-dioxane, and diisopropylamine. For very sensitive reactions, solvents were further treated with metallic sodium, calcium hydride, or molecular sieves, and distilled just prior to use. Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. The symbol ♦ denotes that the chlorine isotope pattern was observed in the mass spectrum.

Chiral separations were used to separate enantiomers or diastereomers of some intermediates during the preparation of the compounds of the invention. When chiral separation was done, the separated enantiomers were designated as ENT-1 or ENT-2 (or DIAST-1 or DIAST-2), according to their order of elution. In some embodiments, enantiomers designated as ENT-1 or ENT-2 can be used as starting materials to prepare other enantiomers or diastereomers. In such situations, the resulting enantiomers prepared are designated as ENT-X1 and ENT-X2, respectively, according to their starting materials; similarly, the diastereomers prepared are designated as DIAST-X1 and DIAST-X2, respectively, (or DIAST-according to their starting materials. DIAST-Y and DIAST-Z nomenclature is used similarly, in syntheses employing multiple intermediates.

Reactions proceeding through detectable intermediates were generally followed by LCMS, and allowed to proceed to full conversion prior to addition of subsequent reagents. For syntheses referencing procedures in other Examples or Methods, reaction conditions (reaction time and temperature) may vary. In general, reactions were followed by thin-layer chromatography or mass spectrometry, and subjected to work-up when appropriate. Purifications may vary between experiments: in general, solvents and the solvent ratios used for eluents/gradients were chosen to provide appropriate R_{f}s or retention times. All starting materials in these Preparations and Examples are either commercially available or can be prepared by methods known in the art or as described herein.

### Preparation P7

### tert-Butyl 4-[2-(5-chloropyridin-2-yl)-2-methyl-1,3-benzodioxol-4-yl]piperidine-1-carboxylate (P7)

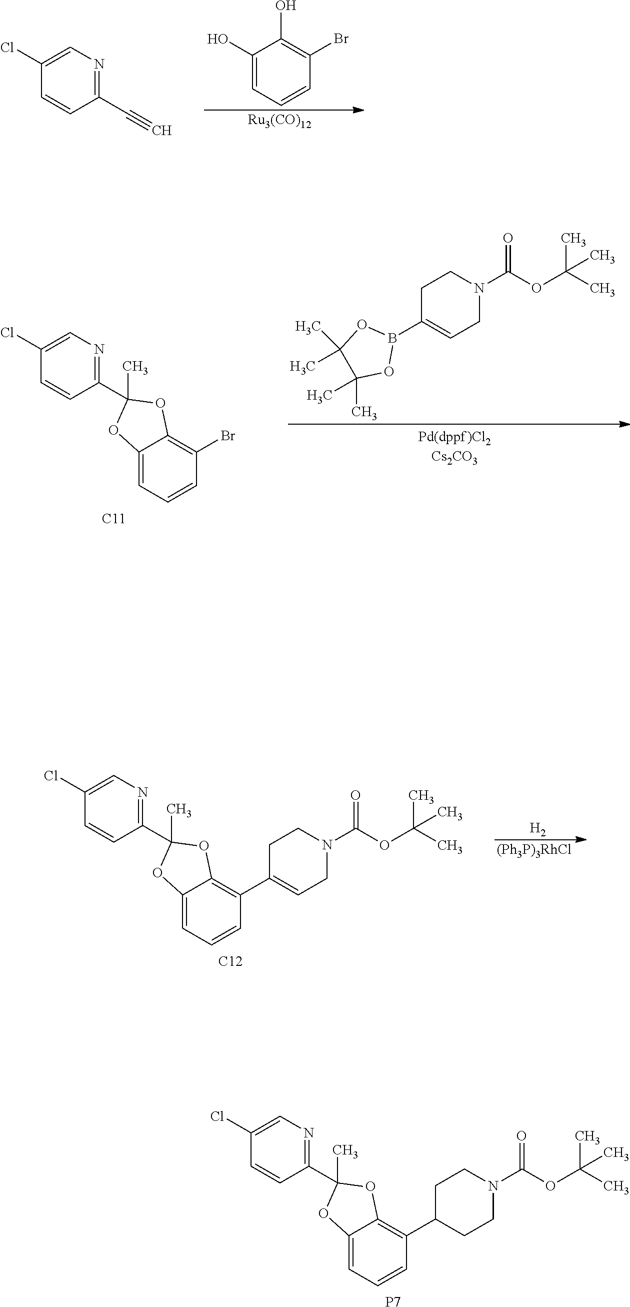

### Step 1. Synthesis of 2-(4-bromo-2-methyl-1,3-benzodioxol-2-yl)-5-chloropyridine (C11)

A mixture of 5-chloro-2-ethynylpyridine (1.80 g, 13.1 mmol), 3-bromobenzene-1,2-diol (2.47 g, 13.1 mmol), and triruthenium dodecacarbonyl (167 mg, 0.261 mmol) in toluene (25 mL) was degassed for 1 minute and then heated at 100° C. for 16 hours. The reaction mixture was diluted with ethyl acetate (30 mL) and filtered through a pad of diatomaceous earth; the filtrate was concentrated in vacuo and purified using silica gel chromatography (Gradient: 0% to 1% ethyl acetate in petroleum ether) to provide C11 as a yellow oil. Yield: 1.73 g, 5.30 mmol, 40%. LCMS m/z 325.6 (bromine-chlorine isotope pattern observed) [M+H]⁺. ¹H NMR (400 MHz, chloroform-d) δ 8.63 (dd, J=2.4, 0.7 Hz, 1H), 7.71 (dd, component of ABX pattern, J=8.4, 2.4 Hz, 1H), 7.60 (dd, component of ABX pattern, J=8.4, 0.7 Hz, 1H), 6.97 (dd, J=8.0, 1.4 Hz, 1H), 6.76 (dd, component of ABX pattern, J=7.8, 1.4 Hz, 1H), 6.72 (dd, component of ABX pattern, J=8.0, 7.8 Hz, 1H), 2.10 (s, 3H).

### Step 2. Synthesis of tert-butyl 4-[2-(5-chloropyridin-2-yl)-2-methyl-1,3-benzodioxol-4-yl]-3,6-dihydropyridine-1(2H)-carboxylate (C12)

[1,1′-Bis(diphenylphosphino)ferrocene]dichloropalladium(II) (388 mg, 0.530 mmol) was added to a suspension of C11 (1.73 g, 5.30 mmol), tert-butyl 4-(4,4,5,5-tetramethyl-1,3,2-dioxaborolan-2-yl)-3,6-dihydropyridine-1(2H)-carboxylate (1.64 g, 5.30 mmol), and cesium carbonate (5.18 g, 15.9 mmol) in 1,4-dioxane (35 mL) and water (6 mL). The reaction mixture was stirred at 90° C. for 4 hours, whereupon it was diluted with ethyl acetate (30 mL) and water (5 mL). The organic layer was concentrated in vacuo and the residue was subjected to silica gel chromatography (Gradient: 0% to 5% ethyl acetate in petroleum ether), affording C12 as a yellow gum. Yield: 1.85 g, 4.31 mmol, 81%. LCMS m/z 451.0♦ [M+Na⁺]. ¹H NMR (400 MHz, chloroform-d) δ 8.62 (dd, J=2.5, 0.8 Hz, 1H), 7.69 (dd, component of ABX pattern, J=8.4, 2.4 Hz, 1H), 7.57 (dd, component of ABX pattern, J=8.4, 0.8 Hz, 1H), 6.84-6.79 (m, 2H), 6.78-6.73 (m, 1H), 6.39-6.33 (br m, 1H), 4.13-4.07 (m, 2H), 3.68-3.58 (m, 2H), 2.60-2.51 (br m, 2H), 2.07 (s, 3H), 1.49 (s, 9H).

### Step 3. Synthesis of tert-butyl 4-[2-(5-chloropyridin-2-yl)-2-methyl-1,3-benzodioxol-4-yl]piperidine-1-carboxylate (P7)

A solution of C12 (2.61 g, 6.08 mmol) and tris(triphenylphosphine)rhodium(I) chloride (Wilkinson's catalyst; 563 mg, 0.608 mmol) in methanol (100 mL) was degassed under vacuum and then purged with hydrogen; this evacuation-purge cycle was carried out a total of three times. The reaction mixture was then stirred at 60° C. under hydrogen (50 psi) for 16 hours, whereupon it was filtered. The filtrate was concentrated in vacuo, and the residue was purified using silica gel chromatography (Gradient: 0% to 10% ethyl acetate in petroleum ether); the resulting material was combined with material from a similar hydrogenation carried out on C12 (110 mg, 0.256 mmol) to provide P7 as a light-yellow gum. Combined yield: 2.05 g, 4.76 mmol, 75%. LCMS m/z 431.3♦ [M+H]⁺. ¹H NMR (400 MHz, chloroform-d) δ 8.62 (d, J=2.3 Hz, 1H), 7.69 (dd, component of ABX pattern, J=8.4, 2.4 Hz, 1H), 7.57 (d, half of AB quartet, J=8.4 Hz, 1H), 6.79 (dd, component of ABC pattern, J=7.8, 7.7 Hz, 1H), 6.72 (dd, component of ABC pattern, J=7.8, 1.3 Hz, 1H), 6.68 (br d, component of ABC pattern, J=7.9 Hz, 1H), 4.32-4.12 (br m, 2H), 2.91-2.73 (m, 3H), 2.05 (s, 3H), 1.90-1.62 (m, 4H), 1.48 (s, 9H).

### Preparations P8 and P9

### tert-Butyl 4-[2-(5-chloropyridin-2-yl)-2-methyl-1,3-benzodioxol-4-yl]piperidine-1-carboxylate, ENT-1 (P8) and tert-Butyl 4-[2-(5-chloropyridin-2-yl)-2-methyl-1,3-benzodioxol-4-yl]piperidine-1-carboxylate, ENT-2 (P9)

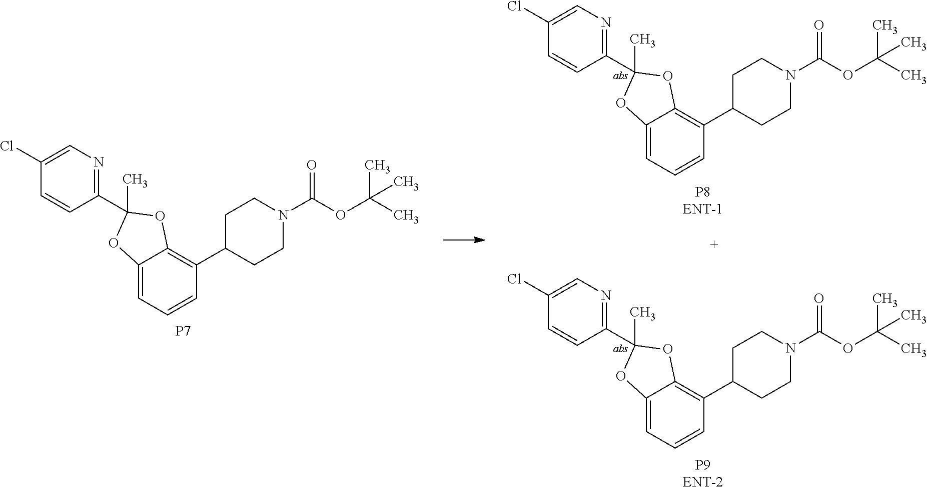

Separation of P7 (500 mg, 1.16 mmol) into its component enantiomers was effected using SFC {Column: Phenomenex Lux Amylose-1, 5 μm; Mobile phase: 9:1 carbon dioxide/[2-propanol containing 0.2% (7 M ammonia in methanol)]}. The first-eluting enantiomer was designated as ENT-1 (P8), and the second-eluting enantiomer as ENT-2 (P9).

P8 Yield: 228 mg, 0.529 mmol, 46%. Retention time 4.00 minutes {Column: Phenomenex Lux Amylose-1, 4.6×250 mm, 5 μm; Mobile phase A: carbon dioxide; Mobile phase B: [2-propanol containing 0.2% (7 M ammonia in methanol)]; Gradient: 5% B for 1.00 minute, then 5% to 60% B over 8.00 minutes; Flow rate: 3.0 mL/minute; Back pressure: 120 bar}.
P9 Yield: 229 mg, 0.531 mmol, 46%. Retention time 4.50 minutes (Analytical conditions identical to those used for P8).

### Preparation P15

### Methyl 2-(chloromethyl)-1-[(2S)-oxetan-2-ylmethyl]-1H-benzimidazole-6-carboxylate (P15)

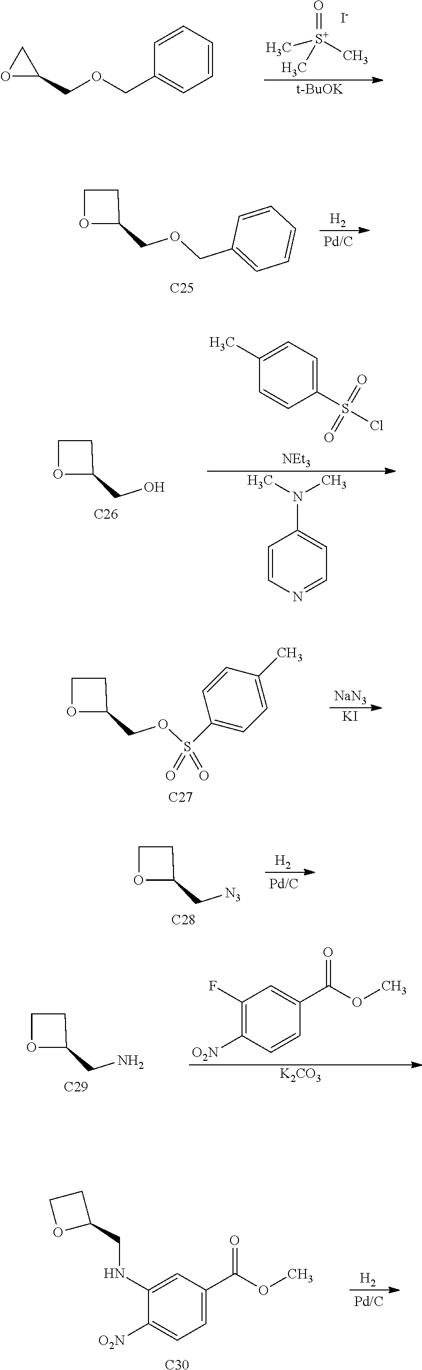

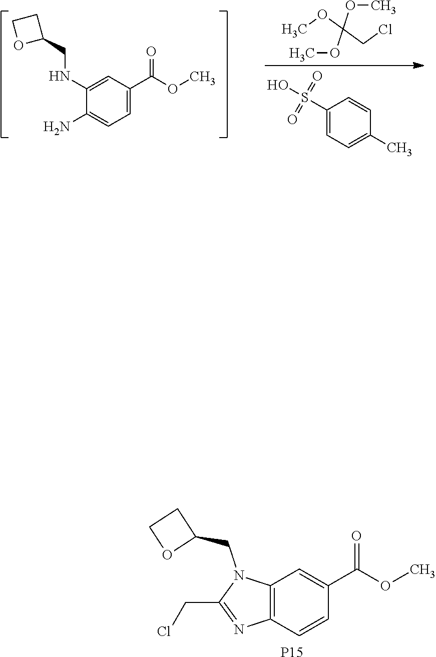

This entire sequence was carried out on large scale. In general, before reactions, as well as after addition of reagents, reactors were evacuated to −0.08 to −0.05 MPa and then filled with nitrogen to normal pressure. This process was generally repeated 3 times, and then oxygen content was assessed to ensure that it was ≤1.0%. For the processes of extraction and washing of organic layers, mixtures were generally stirred for 15 to 60 minutes and then allowed to settle for 15 to 60 minutes before separation of layers.

### Step 1. Synthesis of (2S)-2-[(benzyloxy)methyl]oxetane (C25)

This reaction was carried out in three batches of approximately the same scale. A 2000 L glass-lined reactor was charged with 2-methylpropan-2-ol (774.7 kg). Potassium tert-butoxide (157.3 kg, 1402 mol) was added via a solid addition funnel, and the mixture was stirred for 30 minutes. Trimethylsulfoxonium iodide (308.2 kg, 1400 mol) was then added in the same manner, and the reaction mixture was heated at 55° C. to 65° C. for 2 to 3 hours, whereupon (2S)-2-[(benzyloxy)methyl]oxirane (92.1 kg, 561 mol) was added at a rate of 5 to 20 kg/hour. After the reaction mixture had been maintained at 55° C. to 65° C. for 25 hours, it was cooled to 25° C. to 35° C., and filtered through diatomaceous earth (18.4 kg). The filter cake was rinsed with tert-butyl methyl ether (3×340 kg), and the combined filtrates were transferred to a 5000 L reactor, treated with purified water (921 kg), and stirred for 15 to 30 minutes at 15° C. to 30° C. The organic layer was then washed twice using a solution of sodium chloride (230.4 kg) in purified water (920.5 kg), and concentrated under reduced pressure (≤−0.08 MPa) at ≤45° C. n-Heptane (187 kg) was added, and the resulting mixture was concentrated under reduced pressure (≤−0.08 MPa) at <45° C.; the organic phase was purified using silica gel chromatography (280 kg), with sodium chloride (18.5 kg) on top of the column. The crude material was loaded onto the column using n-heptane (513 kg), and then eluted with a mixture of n-heptane (688.7 kg) and ethyl acetate (64.4 kg). The three batches were combined, providing C25 as an 85% pure light yellow oil (189.7 kg, 906 mmol, 54%). ¹H NMR (400 MHz, chloroform-d), C25 peaks only: δ 7.40-7.32 (m, 4H), 7.32-7.27 (m, 1H), 4.98 (dddd, J=8.1, 6.7, 4.9, 3.7 Hz, 1H), 4.72-4.55 (m, 4H), 3.67 (dd, component of ABX pattern, J=11.0, 4.9 Hz, 1H), 3.62 (dd, component of ABX pattern, J=11.0, 3.7 Hz, 1H), 2.72-2.53 (m, 2H).

### Step 2. Synthesis of (2S)-oxetan-2-ylmethanol (C26)

10% Palladium on carbon (30.7 kg) was added through an addition funnel to a 10° C. to 30° C. solution of 85% pure C25 (from previous step; 185.3 kg, 884.8 mol) in tetrahydrofuran (1270 kg) in a 3000 L stainless steel autoclave reactor. The addition funnel was rinsed with purified water and tetrahydrofuran (143 kg), and the rinses were added to the reaction mixture. After the reactor contents had been purged with nitrogen, they were similarly purged with hydrogen, increasing the pressure to 0.3 to 0.5 MPa and then venting to 0.05 MPa. This hydrogen purge was repeated 5 times, whereupon the hydrogen pressure was increased to 0.3 to 0.4 MPa. The reaction mixture was then heated to 35° C. to 45° C. After 13 hours, during which the hydrogen pressure was maintained at 0.3 to 0.5 MPa, the mixture was vented to 0.05 MPa, and purged five times with nitrogen, via increasing the pressure to 0.15 to 0.2 MPa and then venting to 0.05 MPa. After the mixture had been cooled to 10° C. to 25° C., it was filtered, and the reactor was rinsed with tetrahydrofuran (2×321 kg). The filter cake was soaked twice with this rinsing liquor and then filtered; concentration at reduced pressure (≤−0.06 MPa) was carried out at ≤40° C., affording C26 (62.2 kg, 706 mol, 80%) in tetrahydrofuran (251 kg)

### Step 3. Synthesis of (2S)-oxetan-2-ylmethyl 4-methylbenzenesulfonate (C27)

4-(Dimethylamino)pyridine (17.5 kg, 143 mol) was added to a 10° C. to 25° C. solution of C26 (from the previous step; 62.2 kg, 706 mol) in tetrahydrofuran (251 kg) and triethylamine (92.7 kg, 916 mol) in dichloromethane (1240 kg). After 30 minutes, p-toluenesulfonyl chloride (174.8 kg, 916.9 mol) was added in portions at intervals of 20 to 40 minutes, and the reaction mixture was stirred at 15° C. to 25° C. for 16 hours and 20 minutes. Purified water (190 kg) was added; after stirring, the organic layer was washed with aqueous sodium bicarbonate solution (prepared using 53.8 kg of sodium bicarbonate and 622 kg of purified water), and then washed with aqueous ammonium chloride solution (prepared using 230 kg of ammonium chloride and 624 kg of purified water). After a final wash with purified water (311 kg), the organic layer was filtered through a stainless steel Nutsche filter that had been preloaded with silica gel (60.2 kg). The filter cake was soaked with dichloromethane (311 kg) for 20 minutes, and then filtered; the combined filtrates were concentrated at reduced pressure (≤−0.05 MPa) and ≤40° C. until 330 to 400 L remained. Tetrahydrofuran (311 kg) was then added, at 15° C. to 30° C., and the mixture was concentrated in the same manner, to a final volume of 330 to 400 L. The tetrahydrofuran addition and concentration was repeated, again to a volume of 330 to 400 L, affording a light yellow solution of C27 (167.6 kg, 692 mmol, 98%) in tetrahydrofuran (251.8 kg). ¹H NMR (400 MHz, chloroform-d), C27 peaks only: δ 7.81 (d, J=8.4 Hz, 2H), 7.34 (d, J=8.1 Hz, 2H), 4.91 (ddt, J=8.0, 6.7, 3.9 Hz, 1H), 4.62-4.55 (m, 1H), 4.53-4.45 (m, 1H), 4.14 (d, J=3.9 Hz, 2H), 2.75-2.63 (m, 1H), 2.60-2.49 (m, 1H), 2.44 (s, 3H).

### Step 4. Synthesis of (2S)-2-(azidomethyl)oxetane (C28)

N,N-Dimethylformamide (473 kg), sodium azide (34.7 kg, 534 mol), and potassium iodide (5.2 kg, 31 mol) were combined in a 3000 L glass-lined reactor at 10° C. to 25° C. After addition of C27 (83.5 kg, 344.6 mol) in tetrahydrofuran (125.4 kg), the reaction mixture was heated to 55° C. to 65° C. for 17 hours and 40 minutes, whereupon it was cooled to 25° C. to 35° C., and nitrogen was bubbled from the bottom valve for 15 minutes. tert-Butyl methyl ether (623 kg) and purified water (840 kg) were then added, and the resulting aqueous layer was extracted twice with tert-butyl methyl ether (312 kg and 294 kg). The combined organic layers were washed with purified water (2×419 kg) while maintaining the temperature at 10° C. to 25° C., affording C28 (31.2 kg, 276 mol, 80%) in a solution of the above organic layer (1236.8 kg).

### Step 5. Synthesis of 1-[(2S)-oxetan-2-yl]methanamine (C29)

10% Palladium on carbon (3.7 kg) was added through an addition funnel to a 10° C. to 30° C. solution of C28 [from the previous step; 1264 kg (31.1 kg of C28, 275 mol)] in tetrahydrofuran (328 kg) in a 3000 L stainless steel autoclave reactor. The addition funnel was rinsed with tetrahydrofuran (32 kg), and the rinse was added to the reaction mixture. After the reactor contents had been purged with nitrogen, they were similarly purged with hydrogen, increasing the pressure to 0.05 to 0.15 MPa and then venting to 0.03 to 0.04 MPa. This hydrogen purge was repeated 5 times, whereupon the hydrogen pressure was increased to 0.05 to 0.07 MPa. The reaction temperature was increased to 25° C. to 33° C., and the hydrogen pressure was maintained at 0.05 to 0.15 MPa for 22 hours, while exchanging the hydrogen every 3 to 5 hours. The mixture was then purged five times with nitrogen, via increasing the pressure to 0.15 to 0.2 MPa and then venting to 0.05 MPa. After filtration, tetrahydrofuran (92 kg and 93 kg) was used to wash the reactor and then soak the filter cake. The combined filtrates were concentrated at reduced pressure (≤−0.07 MPa) and ≤45° C., affording C29 (18.0 kg, 207 mol, 75%) in tetrahydrofuran (57.8 kg). ¹H NMR (400 MHz, DMSO-d₆), C29 peaks only: δ 4.62 (ddt, J=7.6, 6.6, 5.1 Hz, 1H), 4.49 (ddd, J=8.6, 7.3, 5.6 Hz, 1H), 4.37 (dt, J=9.1, 5.9 Hz, 1H), 2.69 (d, J=5.1 Hz, 2H), 2.55-2.49 (m, 1H), 2.39 (m, 1H).

### Step 6. Synthesis of methyl 4-nitro-3-{[(2S)-oxetan-2-ylmethyl]amino}benzoate (C30)

Potassium carbonate (58.1 kg, 420 mol) was added to a solution of methyl 3-fluoro-4-nitrobenzoate (54.8 kg, 275 mol) in tetrahydrofuran (148 kg) in a 100 L glass-lined reactor, and the mixture was stirred for 10 minutes. A solution of C29 (29.3 kg, 336 mol) in tetrahydrofuran (212.9 kg) was added, and the reaction mixture was stirred at 20° C. to 30° C. for 12 hours, whereupon ethyl acetate (151 kg) was added, and the mixture was filtered through silica gel (29 kg). The filter cake was rinsed with ethyl acetate (150 kg and 151 kg), and the combined filtrates were concentrated at reduced pressure (≤−0.08 MPa) and ≤45° C. to a volume of 222 to 281 L. After the mixture had been cooled to 10° C. to 30° C., n-heptane (189 kg) was added, stirring was carried out for 20 minutes, and the mixture was concentrated at reduced pressure (≤−0.08 MPa) and ≤45° C. to a volume of 222 L. n-Heptane (181 kg) was again added into the mixture at a reference rate of 100 to 300 kg/hour, and stirring was continued for 20 minutes. The mixture was sampled until residual tetrahydrofuran was ≤5% and residual ethyl acetate was 10% to 13%. The mixture was heated to 40° C. to 45° C. and stirred for 1 hour, whereupon it was cooled to 15° C. to 25° C. at a rate of 5° C. to 10° C. per hour, and then stirred at 15° C. to 25° C. for 1 hour. Filtration using a stainless steel centrifuge provided a filter cake, which was rinsed with a mixture of ethyl acetate (5.0 kg) and n-heptane (34 kg), and then stirred with tetrahydrofuran (724 kg) at 10° C. to 30° C. for 15 minutes; filtration provided a yellow solid largely composed of C30 (57.3 kg, 210 mol, 76%). ¹H NMR (400 MHz, DMSO-d₆) 8.34 (t, J=5.8 Hz, 1H), 8.14 (d, J=8.9 Hz, 1H), 7.63 (d, J=1.7 Hz, 1H), 7.13 (dd, J=8.9, 1.8 Hz, 1H), 4.99 (dddd, J=7.7, 6.7, 5.3, 4.1 Hz, 1H), 4.55 (ddd, J=8.6, 7.3, 5.8 Hz, 1H), 4.43 (dt, J=9.1, 6.0 Hz, 1H), 3.87 (s, 3H), 3.67-3.61 (m, 2H), 2.67 (dddd, J=11.1, 8.6, 7.7, 6.2 Hz, 1H), 2.57-2.47 (m, 1H).

### Step 7. Synthesis of methyl 2-(chloromethyl)-1-[(2S)-oxetan-2-ylmethyl]-1H-benzimidazole-6-carboxylate (P15)

A solution of C30 (from the previous step; 51.8 kg, 190 mol) in tetrahydrofuran (678 kg), in a 3000 L autoclave reactor, was treated with 10% palladium on carbon (5.2 kg) at 10° C. to 30° C. The addition pipe was rinsed with tetrahydrofuran (46 kg) and the rinse was added to the reaction mixture. After the reactor contents had been purged with nitrogen, they were similarly purged with hydrogen, increasing the pressure to 0.1 to 0.2 MPa and then venting to 0.02 to 0.05 MPa. This hydrogen purge was repeated 5 times, whereupon the hydrogen pressure was increased to 0.1 to 0.25 MPa. The reaction mixture was stirred at 20° C. to 30° C., and every 2 to 3 hours, the mixture was purged with nitrogen three times, and then purged with hydrogen five times; after each final hydrogen exchange, the hydrogen pressure was increased to 0.1 to 0.25 MPa. After 11.25 hours total reaction time, the reaction mixture was vented to normal pressure, and purged five times with nitrogen, via increasing the pressure to 0.15 to 0.2 MPa and then venting to 0.05 MPa. It was then filtered, and the filter cake was rinsed twice with tetrahydrofuran (64 kg and 63 kg); the combined rinse and filtrate were concentrated under reduced pressure (≤−0.08 MPa) and ≤40° C. to a volume of 128 to 160 L. Tetrahydrofuran (169 kg) was added, and the mixture was again concentrated to a volume of 128 to 160 L; this process was repeated a total of 4 times, affording a solution of the intermediate methyl 4-amino-3-{[(2S)-oxetan-2-ylmethyl]amino}benzoate.

Tetrahydrofuran (150 kg) was added to this solution, followed by 2-chloro-1,1,1-trimethoxyethane (35.1 kg, 227 mol) and p-toluenesulfonic acid monohydrate (1.8 kg, 9.5 mol). After the reaction mixture had been stirred for 25 minutes, it was heated at 40° C. to 45° C. for 5 hours, whereupon it was concentrated under reduced pressure to a volume of 135 to 181 L. 2-Propanol (142 kg) was added, and the mixture was again concentrated to a volume of 135 to 181 L, whereupon 2-propanol (36.5 kg) and purified water (90 kg) were added, and stirring was continued until a solution was obtained. This was filtered with an in-line liquid filter, and then treated with purified water (447 kg) at a reference rate of 150 to 400 kg/hour at 20° C. to 40° C. After the mixture had been cooled to 20° C. to 30° C., it was stirred for 2 hours, and the solid was collected via filtration with a centrifuge. The filter cake was rinsed with a solution of 2-propanol (20.5 kg) and purified water (154 kg); after drying, P15 was obtained as a white solid (32.1 kg, 109 mol, 57%). ¹H NMR (400 MHz, chloroform-d) δ 8.14-8.11 (m, 1H), 8.01 (dd, J=8.5, 1.1 Hz, 1H), 7.79 (br d, J=8.6 Hz, 1H), 5.26-5.18 (m, 1H), 5.04 (s, 2H), 4.66-4.58 (m, 2H), 4.53 (dd, component of ABX pattern, J=15.7, 2.7 Hz, 1H), 4.34 (dt, J=9.1, 6.0 Hz, 1H), 3.96 (s, 3H), 2.82-2.71 (m, 1H), 2.48-2.37 (m, 1H).

Alternatively, P15 can be prepared using the methods described in U.S. Pat. No. 10,208,019 (see Intermediate 23 at Column 58 of the patent), which is hereby incorporated by reference in its entirety.

### Example 1

### 2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2 (Compound 1) [from P9]

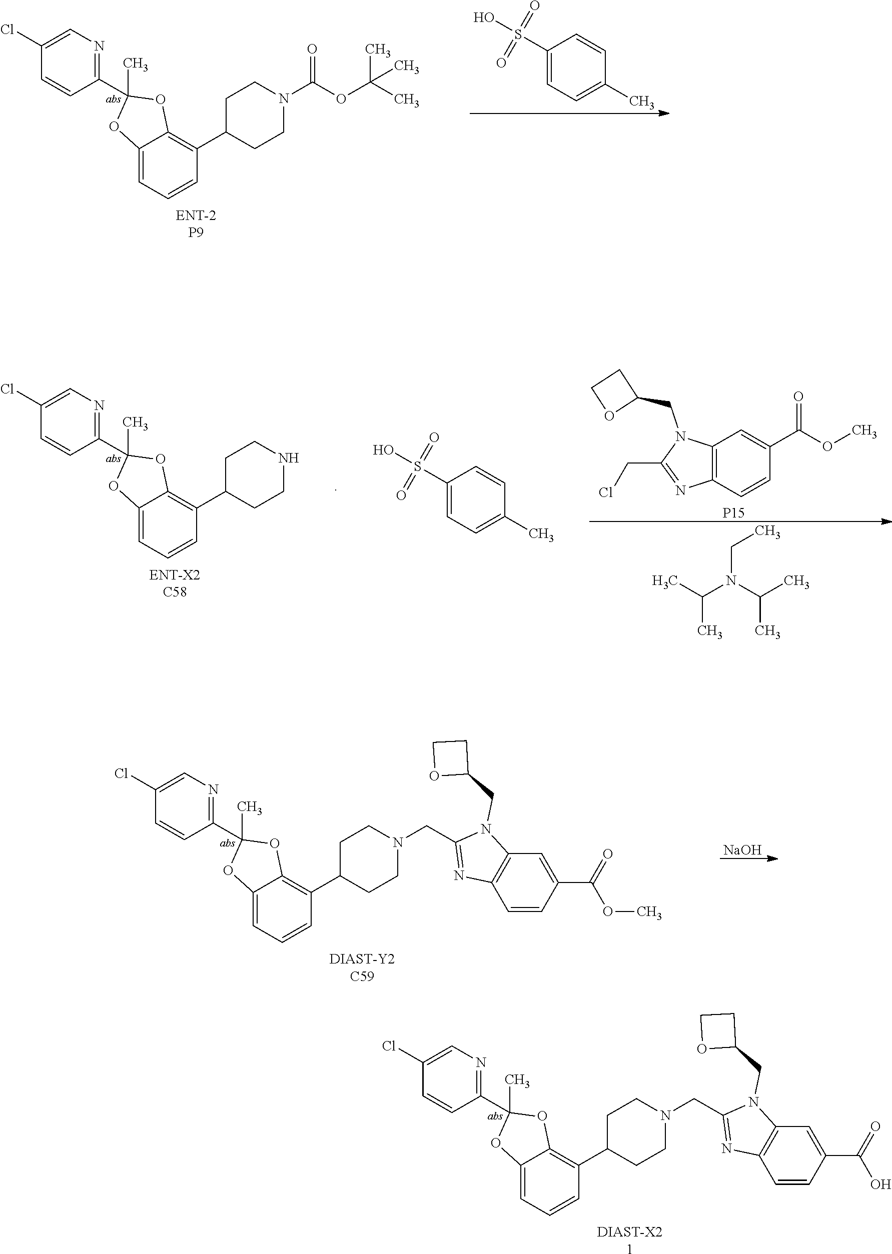

### Step 1. Synthesis of 5-chloro-2-[2-methyl-4-(piperidin-4-yl)-1,3-benzodioxol-2-yl]pyridine, ENT-X2, p-toluenesulfonate salt (C58) [from P9]

A solution of P9 (228 mg, 0.529 mmol) in ethyl acetate (2.7 mL) was treated with p-toluenesulfonic acid monohydrate (116 mg, 0.610 mmol), and the reaction mixture was heated at 50° C. for 16 hours. It was then allowed to stir at room temperature overnight, whereupon the precipitate was collected via filtration and rinsed with a mixture of ethyl acetate and heptane (1:1, 2×20 mL) to provide C58 as a white solid. Yield: 227 mg, 0.451 mmol, 85%. LCMS m/z 331.0♦ [M+H]⁺. ¹H NMR (400 MHz, DMSO-d₆): δ 8.73 (d, J=2.4 Hz, 1H), 8.61-8.46 (br m, 1H), 8.35-8.18 (br m, 1H), 8.02 (dd, J=8.5, 2.5 Hz, 1H), 7.64 (d, J=8.5 Hz, 1H), 7.47 (d, J=7.8, 2H), 7.11 (d, J=7.8 Hz, 2H), 6.89-6.81 (m, 2H), 6.72 (pentet, J=4.0 Hz, 1H), 3.45-3.27 (m, 2H, assumed; partially obscured by water peak), 3.10-2.91 (m, 3H), 2.28 (s, 3H), 2.02 (s, 3H), 1.97-1.80 (m, 4H).

### Step 2. Synthesis of methyl 2-({4-[2-(5-chloropyridin-2-yl)-2-methyl-1,3-benzodioxol-4-yl]piperidin-1-yl}methyl)-1-[(2S)-oxetan-2-ylmethyl]-1H-benzimidazole-6-carboxylate, DIAST-Y2 (C59) [from P9]

N,N-Diisopropylethylamine (0.234 mL, 1.34 mmol) was added to a solution of C58 (225 mg, 0.447 mmol) in acetonitrile (2.2 mL). After this mixture had been stirred for 5 minutes at 45° C., P15 (120 mg, 0.407 mmol) was added, and stirring was continued at 45° C. for 16 hours, whereupon P15 (11 mg, 37 μmol) was again added. After an additional 3 hours of stirring, the reaction mixture was treated with water (2.5 mL) and allowed to cool to room temperature. More water (5 mL) was added, and the resulting slurry was stirred for 2 hours, whereupon the solid was collected via filtration and washed with a mixture of acetonitrile and water (15:85, 3×5 mL) to afford C59 as an off-white solid (252 mg). This material contained some N,N-diisopropylethylamine by ¹H NMR analysis, and was taken directly to the following step. LCMS m/z 589.1♦ [M+H]⁺. ¹H NMR (400 MHz, chloroform-d) 8.61 (d, J=2.3 Hz, 1H), 8.18 (d, J=1.5 Hz, 1H), 7.96 (dd, J=8.5, 1.5 Hz, 1H), 7.74 (d, J=8.5 Hz, 1H), 7.67 (dd, component of ABX pattern, J=8.4, 2.4 Hz, 1H), 7.59-7.51 (m, 1H), 6.82-6.75 (m, 1H), 6.74-6.66 (m, 2H), 5.28-5.19 (m, 1H), 4.75 (dd, component of ABX pattern, J=15.3, 6.0 Hz, 1H), 4.68 (dd, component of ABX pattern, J=15.3, 3.4 Hz, 1H), 4.67-4.58 (m, 1H), 4.41 (ddd, J=9.1, 5.9, 5.9 Hz, 1H), 3.95 (s, 2H), 3.95 (s, 3H), 3.07-2.89 (m, 2H), 2.81-2.69 (m, 2H), 2.53-2.41 (m, 1H), 2.37-2.22 (m, 2H), 2.05 (s, 3H), 1.93-1.74 (m, 4H).

### Step 3. Synthesis of 2-({4-[2-(5-chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2 (Compound 1) [from P9]

A suspension of C59 (from the previous step; 250 mg, 50.407 mmol) in methanol (2 mL) was heated to 40° C., whereupon aqueous sodium hydroxide solution (1 M; 0.81 mL, 0.81 mmol) was added. After 17 hours, the reaction mixture was allowed to cool to room temperature, and the pH was adjusted to 5 to 6 with 1 M aqueous citric acid solution. The resulting mixture was diluted with water (2 mL), stirred for 2 hours, and extracted with ethyl acetate (3×5 mL); the combined organic layers were washed with saturated aqueous sodium chloride solution (5 mL), dried over sodium sulfate, filtered, and concentrated in vacuo to provide a foamy solid. This material was taken up in a mixture of ethyl acetate and heptane (1:1, 4 mL), heated to 50° C., and then allowed to cool and stir overnight. Filtration afforded Compound 1 as a white solid. Yield: 179 mg, 0.311 mmol, 76% over 2 steps. LCMS m/z 575.1♦ [M+H]⁺. ¹H NMR (400 MHz, DMSO-d₆) δ 12.73 (br s, 1H), 8.71 (d, J=2.5 Hz, 1H), 8.27 (d, J=1.5 Hz, 1H), 8.00 (dd, J=8.5, 2.5 Hz, 1H), 7.80 (dd, J=8.4, 1.6 Hz, 1H), 7.64 (d, J=8.4 Hz, 1H), 7.60 (d, J=8.5 Hz, 1H), 6.83-6.72 (m, 3H), 5.14-5.06 (m, 1H), 4.77 (dd, component of ABX pattern, J=15.2, 7.2 Hz, 1H), 4.63 (dd, component of ABX pattern, J=15.2, 2.8 Hz, 1H), 4.50-4.42 (m, 1H), 4.37 (ddd, J=9.0, 5.9, 5.9 Hz, 1H), 3.85 (AB quartet, J_{AB}=13.6 Hz, Δ□_{AB}=71.5 Hz, 2H), 3.01 (br d, J=11.2 Hz, 1H), 2.85 (br d, J=11.2 Hz, 1H), 2.74-2.57 (m, 2H), 2.47-2.38 (m, 1H), 2.29-2.10 (m, 2H), 2.01 (s, 3H), 1.81-1.64 (m, 4H).

### Synthesis 1S-1. Synthesis of Compound 1, 1,3-dihydroxy-2-(hydroxymethyl)propan-2-aminium salt

### Synthesis of 1,3-dihydroxy-2-(hydroxymethyl)propan-2-aminium 2-({4-[2-(5-chloropyridin-2-yl)-2-methyl-1,3-benzodioxol-4-yl]piperidin-1-yl}methyl)-1-[(2S)-oxetan-2-ylmethyl]-1H-benzimidazole-6-carboxylate, DIAST-X2 (Compound 1, 1,3-dihydroxy-2-(hydroxymethyl)propan-2-aminium salt) [from P9]

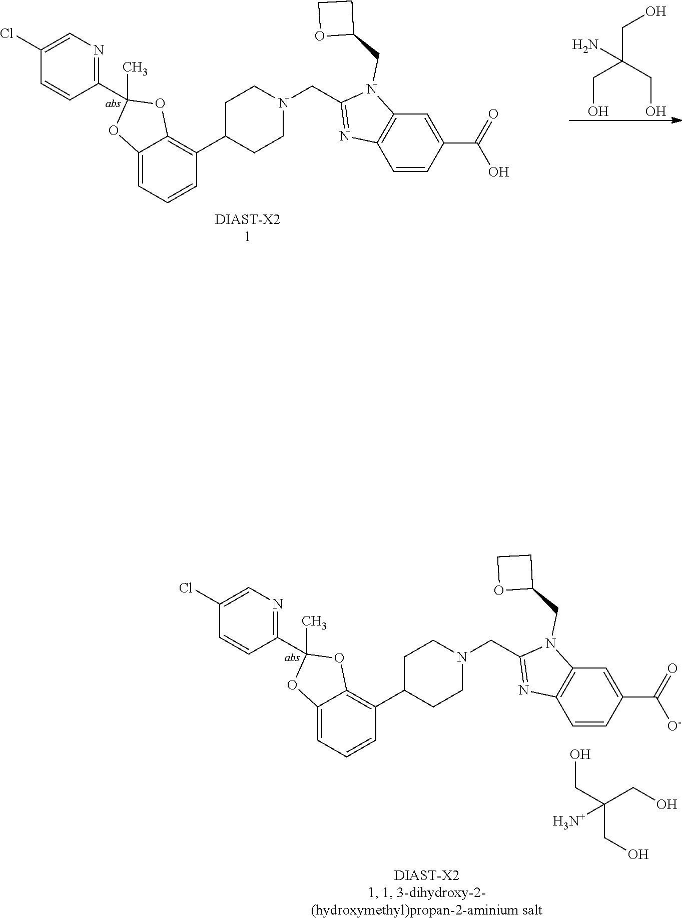 [SMILES: CC1(c2ccc(Cl)cn2)Oc2cccc(C3CCN(Cc4nc5ccc(C(=O)O)cc5n4C[C@@H]4CCO4)CC3)c2O1 ; NC(CO)(CO)CO ; "DIAST-X21" ; CC1(c2ccc(Cl)cn2)Oc2cccc(C3CCN(Cc4nc5ccc(C(=O)[O-])cc5n4C[C@@H]4CCO4)CC3)c2O1 ; [NH3+]C(CO)(CO)CO ; "DIAST-X21, 1, 3-dihydroxy-2-(hydroxymethyl)propan-2-aminium salt"]

A mixture of Compound 1 (1.54 g, 2.68 mmol) in tetrahydrofuran (10 mL) was treated with an aqueous solution of 2-amino-2-(hydroxymethyl)propane-1,3-diol (Tris, 1.0 M; 2.81 mL, 2.81 mmol). After 24 hours, the reaction mixture was concentrated in vacuo with ethanol (2×50 mL). The residue was treated with ethanol (15 mL). After stirring for 20 hours, the solid was collected via filtration and washed with cold ethanol (5 mL) to afford Compound 1, 1,3-dihydroxy-2-(hydroxymethyl)propan-2-aminium salt as a white solid. Yield: 1.41 g, 2.03 mmol, 76%. LCMS m/z 575.3♦ [M+H]⁺. ¹H NMR (600 MHz, DMSO-d₆) δ 8.71 (d, J=2.5 Hz, 1H), 8.21 (br s, 1H), 8.00 (dd, J=8.5, 2.5 Hz, 1H), 7.79 (br d, J=8.4 Hz, 1H), 7.60 (d, J=8.5 Hz, 1H), 7.57 (d, J=8.4 Hz, 1H), 6.82-6.73 (m, 3H), 5.13-5.07 (m, 1H), 4.74 (dd, J=15.3, 7.2 Hz, 1H), 4.61 (dd, J=15.3, 2.9 Hz, 1H), 4.49-4.43 (m, 1H), 4.37 (ddd, J=9.0, 5.9, 5.9 Hz, 1H), 3.93 (d, J=13.6 Hz, 1H), 3.75 (d, J=13.5 Hz, 1H), 3.01 (br d, J=11.3 Hz, 1H), 2.86 (br d, J=11.4 Hz, 1H), 2.73-2.59 (m, 2H), 2.48-2.37 (m, 1H), 2.27-2.20 (m, 1H), 2.19-2.12 (m, 1H), 2.01 (s, 3H), 1.82-1.66 (m, 4H). mp=184° C. to 190° C.

### Synthesis 1S-2. Alternative Synthesis of Compound 1, 1,3-dihydroxy-2-(hydroxymethyl)propan-2-aminium salt

A mixture of Compound 1 (8.80 gm, 15.3 mmol) in 2-methyltetrahydrofuran (90 ml) was concentrated in vacuo on a rotary evaporator, in a 37° C. water bath, to reduce the total volume to ˜54 ml. Isopropanol (90 ml) was added to the mixture and then again concentrate the resulting mixture to a volume of ˜54 ml. Isopropanol (135 ml) was added to the mixture, followed by addition of aqueous tris amine (3M; 5.0 ml, 0.98 equiv). The resulting mixture/solution was stirred at ambient temperature; and a solid precipitate began to form within ˜15 min. The mixture was then stirred at ambient temperature for additional 5 hr. The resulting mixture/slurry was cooled to 0° C. and the cooled slurry was stirred for about another 2 hr. The slurry was filtered and washed with cold isopropanol (3×15 ml). The solid collected was allowed to Air dry on the collection funnel for about 90 min and then transfer to the vacuum oven for overnight drying. After ˜16 hr at 50° C./23 in Hg vacuum (with a slight nitrogen bleed) 8.66 gm of Compound 1, 1,3-dihydroxy-2-(hydroxymethyl)propan-2-aminium salt was obtained as white solid; 99.8 area % by UPLC (yield:12.5 mmol, 81%). LCMS and ¹H NMR data were obtained, which are substantially the same as those in Synthesis 1S-1 shown above.

Acquisition of Powder X-Ray Diffraction (PXRD) Data for Form A of Compound 1, 1,3-dihydroxy-2-(hydroxymethyl)propan-2-aminium salt (Also Known as Form A of Anhydrous Tris Salt of Compound 1)

The white solid of the tris salt of Compound 1 (from both Synthesis 1S-1 and Synthesis 1S-2) was submitted for PXRD analysis and found to be a crystalline material (which is designated as Form A). Powder X-ray diffraction analysis was conducted using a Bruker AXS D8 Endeavor diffractometer equipped with a Cu radiation source. The divergence slit was set at 15 mm continuous illumination. Diffracted radiation was detected by a PSD-Lynx Eye detector, with the detector PSD opening set at 2.99 degrees. The X-ray tube voltage and amperage were set to 40 kV and 40 mA respectively. Data was collected at the Cu wavelength (CuK_{α}=1.5418λ) in the Theta-Theta goniometer from 3.0 to 40.0 degrees 2-Theta using a step size of 0.01 degrees and a step time of 1.0 second. The antiscatter screen was set to a fixed distance of 1.5 mm. Samples were rotated during data collection. Samples were prepared by placing them in a silicon low background sample holder and rotated during collection. Data were collected using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity of ≥3% were generally chosen. Typically, the peaks which were not resolved or were consistent with noise were not selected. A typical error associated with the peak position from PXRD stated in USP up to +/−0.2° 2-Theta (USP-941). A list of diffraction peaks expressed in terms of the degree 2θ and relative intensities with a relative intensity of ≥3.0% of a PXRD from a sample of Form A obtained by Synthesis 1S-2 is provided above in Table E1-1.

TABLE E1-1

| Angle (2 theta) +/− 0.2° | Relative Intensity (%) |
|---|---|
| 3.9 | 18.4 |
| 7.7 | 36.3 |
| 8.1 | 10.4 |
| 8.7 | 3.4 |
| 10.2 | 4.1 |
| 14.6 | 5.8 |
| 15.2 | 30.1 |
| 15.7 | 45.5 |
| 16.0 | 31.3 |
| 16.8 | 8.7 |
| 17.6 | 86.0 |
| 19.2 | 46.6 |
| 19.5 | 25.4 |
| 19.8 | 31.4 |
| 20.2 | 25.0 |
| 21.1 | 100.0 |
| 21.4 | 40.2 |
| 22.2 | 37.0 |
| 23.0 | 19.8 |
| 24.3 | 43.0 |
| 25.0 | 9.9 |
| 26.0 | 15.8 |
| 27.3 | 35.3 |
| 28.2 | 14.1 |
| 29.3 | 19.7 |
| 29.8 | 11.7 |
| 31.6 | 9.3 |
| 32.8 | 6.0 |
| 34.0 | 14.4 |
| 34.5 | 12.1 |
| 35.4 | 3.0 |
| 36.5 | 4.1 |

The anhydrous (anhydrate) crystalline form of tris salt of Compound 1 obtained by the methods described herein is designated as Form A. Form A can be identified by its unique solid state signatures with respect to, for example, powder X-ray diffraction pattern (PXRD), and other solid state methods such as ¹³C solid state NMR.

In some embodiments, Form A exhibits a powder X-ray diffraction pattern comprising at least two characteristic peaks, in terms of 2θ, selected from at 7.7±0.2°; 15.2±0.2°; 15.7±0.2°; and 17.6±0.2°. In some embodiments, Form A exhibits a powder X-ray diffraction pattern comprising at least three characteristic peaks, in terms of 2θ, selected from at 7.7±0.2°; 15.2±0.2°; 15.7±0.2°; and 17.6±0.2°. In some embodiments, Form A exhibits a powder X-ray diffraction pattern comprising characteristic peaks, in terms of 2θ, selected from at 7.7±0.2°; 15.2±0.2°; 15.7±0.2°; and 17.6±0.2°.

In some embodiments, Form A exhibits a powder X-ray diffraction pattern comprising characteristic peaks, in terms of 2θ, at 7.7±0.2° and 17.6±0.2°.

In some embodiments, Form A exhibits a powder X-ray diffraction pattern comprising peaks, in terms of 2θ, at 7.7±0.2°; 15.2±0.2°; and 17.6±0.2°.

In some embodiments, Form A exhibits a powder X-ray diffraction pattern comprising peaks, in terms of 2θ, at 7.7±0.2°; 15.2±0.2°; and 15.7±0.2°.

In some embodiments, Form A exhibits a powder X-ray diffraction pattern comprising peaks, in terms of 2θ, at 7.7±0.2°; 15.2±0.2°; 15.7+0.2°; and 17.6±0.2°.

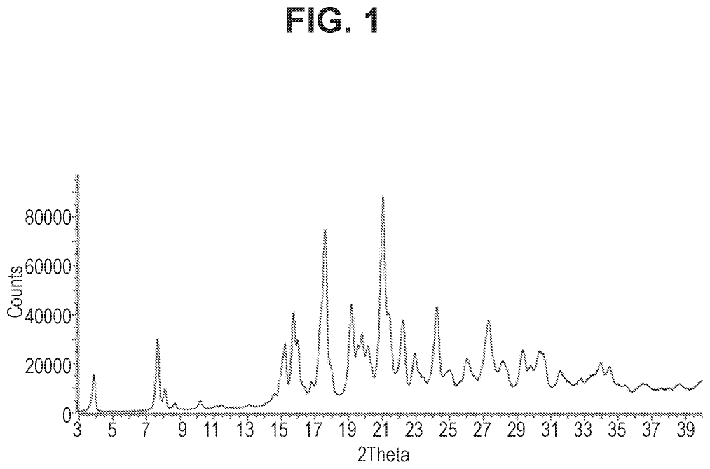

In some embodiments, Form A exhibits a powder X-ray diffraction pattern substantially as shown in FIG. 1.

### Solid State NMR Analysis of Form A of 1,3-Dihydroxy-2-(hydroxymethyl)propan-2-aminium Salt of Compound 1

Solid state NMR (ssNMR) analysis was conducted on a CPMAS probe positioned into a Bruker-BioSpin Avance III 500 MHz (¹H frequency) NMR spectrometer. A sample of Form A of 1,3-Dihydroxy-2-(hydroxymethyl)propan-2-aminium Salt of Compound 1 was packed into a 4 mm rotor. A magic angle spinning rate of 15.0 kHz was used.

¹³C ssNMR spectrum was collected using a proton decoupled cross-polarization magic angle spinning (CPMAS) experiment. A phase modulated proton decoupling field of 80-90 kHz was applied during spectral acquisition. The cross-polarization contact time was set to 2 ms and the recycle delay of 3-8 seconds. The number of scans was adjusted to obtain an adequate signal to noise ratio, with 2048 scans being collected for each API. The ¹³C chemical shift scale was referenced using a ¹³C CPMAS experiment on an external standard of crystalline adamantane, setting its up-field resonance to 29.5 ppm.

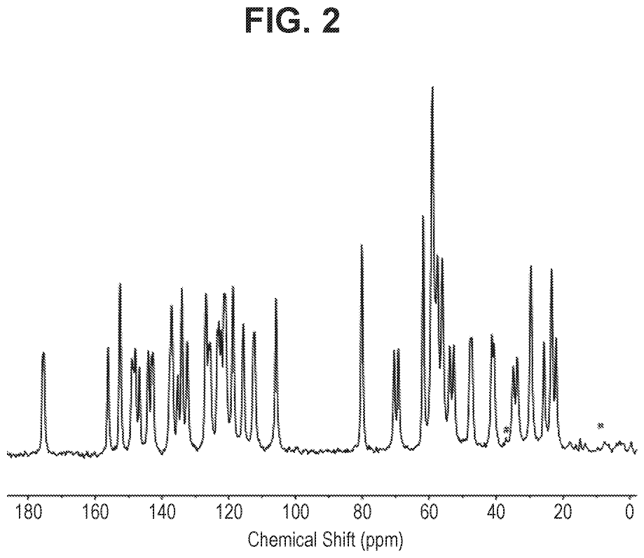

Automatic peak picking was performed using Bruker-BioSpin TopSpin version 3.6 software. Generally, a threshold value of 3% relative intensity was used for preliminary peak selection. The output of the automated peak picking was visually checked to ensure validity and adjustments were manually made if necessary. Although specific solid state NMR peak (ssNMR) values are reported herein there does exist a range for these peak values due to differences in instruments, samples, and sample preparation. This is common practice in the art of ssNMR because of the variation inherent in peak positions. A typical variability for a ¹³C chemical shift x-axis value is on the order of plus or minus 0.2 ppm for a crystalline solid. The ssNMR peak heights reported herein are relative intensities. Solid state NMR intensities can vary depending on the actual setup of the CPMAS experimental parameters and the thermal history of the sample. The chemical shift data is dependent on the testing conditions (i.e. spinning speed and sample holder), reference material, and data processing parameters, among other factors. Typically, the ss-NMR results are accurate to within about ±0.2 ppm. A representative ¹³C ssNMR spectrum of Form A was obtained, which is shown in FIG. 2. ¹³C Chemical Shifts [ppm]±0.2 ppm of Form A are listed in Table E1-2.

TABLE E1-2
Carbon chemical shifts observed.

| ¹³C Chemical Shifts [ppm] ± 0.2 ppm | Relative Intensity |
|---|---|
| 22.1 | 30 |
| 23.4 | 49 |
| 25.7 | 29 |
| 29.6 | 50 |
| 33.7 | 25 |
| 34.9 | 22 |
| 40.7 | 29 |
| 41.3 | 31 |
| 47.3 | 31 |
| 47.7 | 30 |
| 52.7 | 28 |
| 53.8 | 28 |
| 56.1 | 53 |
| 57.5 | 53 |
| 59.1 | 100 |
| 61.8 | 64 |
| 69.2 | 28 |
| 70.5 | 27 |
| 80.1 | 57 |
| 105.9 | 42 |
| 112.4 | 33 |
| 112.5 | 33 |
| 115.6 | 35 |
| 118.7 | 46 |
| 121.1 | 44 |
| 121.4 | 44 |
| 122.3 | 34 |
| 123.0 | 36 |
| 123.4 | 34 |
| 125.6 | 30 |
| 126.0 | 30 |
| 126.8 | 44 |
| 132.4 | 31 |
| 134.1 | 46 |
| 135.2 | 21 |
| 137.1 | 41 |
| 137.6 | 26 |
| 142.6 | 28 |
| 143.1 | 27 |
| 144.0 | 28 |
| 146.7 | 23 |
| 147.9 | 29 |
| 148.5 | 23 |
| 148.9 | 26 |
| 152.5 | 47 |
| 156.1 | 29 |
| 175.3 | 28 |
| 175.6 | 27 |

### Example 2

### Form 2 of 1,3-dihydroxy-2-(hydroxymethyl)propan-2-aminium salt of Compound 1

### Preparation of Form 2 of 1,3-dihydroxy-2-(hydroxymethyl)propan-2-aminium salt of Compound 1

Compound 1 (49.7 mg) was mixed with methanol (0.828 mL) in a vial and heated to 50° C. A stock solution of Tris (30.25 μL, 3M) was then added and the resultant mixture was cooled to room temperature slowly. The mixture was then allowed to slowly evaporate at room temperature (the vial was placed in a fume hood and the cap slightly cracked to allow for solvent evaporation). Crystals of tris salt of Compound 1 formed by slow evaporation in the methanol/water mixed solvent (and this crystalline form is designated as Form 2).

### Single Crystal X-Ray Analysis.

A sample of Form 2 of tris salt of Compound 1 was tested for single crystal analysis. Data collection was performed on a Bruker D8 Venture diffractometer at room temperature. Data collection consisted of omega and phi scans.

The structure was solved by intrinsic phasing using SHELX software suite in the Monoclinic class space group P2₁. The structure was subsequently refined by the full-matrix least squares method. All non-hydrogen atoms were found and refined using anisotropic displacement parameters.

Terminal ring (C1-C2-C3-C4-C5-Cl1) was disordered. A disorder model was tested for this group, but did not refine satisfactorily. CIF_Check module generated level “A” based on above mentioned segment.

The hydrogen atoms located on nitrogen and oxygen were found from the Fourier difference map and refined with distances restrained. The remaining hydrogen atoms were placed in calculated positions and were allowed to ride on their carrier atoms. The final refinement included isotropic displacement parameters for all hydrogen atoms.

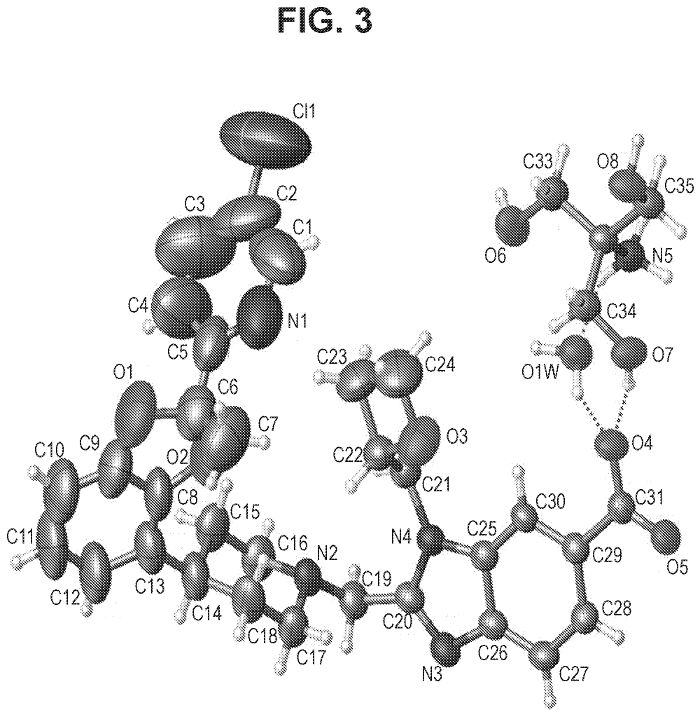

TRIS salt was confirmed because of proton transfer from O5 to N5. Additionally, the structure contained one water molecule (and thus monohydrate). Analysis of the absolute structure using likelihood methods (Hooft 2008) was performed using PLATON (Spek 2010), with the known stereochemistry information of C22 (and thus, the stereochemistry information of C6 was determined). The refined structure was plotted using the SHELXTL plotting package (FIG. 3). According to the refined structure, Form 2 is a monohydrate of tris salt of Compound 1, the structure of which can be represented as shown below:

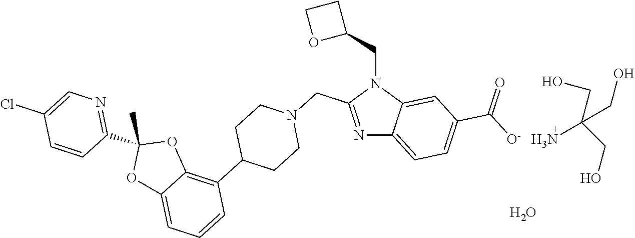 [SMILES: C[C@]1(c2ccc(Cl)cn2)Oc2cccc(C3CCN(Cc4nc5ccc(C(=O)[O-])cc5n4C[C@@H]4CCO4)CC3)c2O1 ; O ; NC(CO)(CO)CO]

The final R-index was 6.6%. A final difference Fourier revealed no missing or misplaced electron density.

Pertinent crystal, data collection and refinement are summarized in Table E2-1. Atomic coordinates, bond lengths, bond angles and displacement parameters are listed in tables E2-2 to E2-4.

TABLE E2-1
Crystal data and structure refinement for Form 2.

| Empirical formula | C35 H44 Cl N5 O9 | |
|---|---|---|
| Formula weight | 714.20 | |
| Temperature | 296(2) K | |
| Wavelength | 1.54178 Å | |
| Crystal system | Monoclinic | |
| Space group | P2₁ | |
| Unit cell dimensions | a = 12.944(4) Å | α = 90°. |
| | b = 6.1938(16) Å | β = 91.731(16)°. |
| | c = 24.777(7) Å | γ = 90°. |

| | |
|---|---|
| Volume | 1985.5(9) Å³ |
| Z | 2 |
| Density (calculated) | 1.195 Mg/m³ |
| Absorption coefficient | 1.311 mm⁻¹ |
| F(000) | 756 |
| Crystal size | 0.500 × 0.060 × 0.020 mm³ |
| Theta range for data collection | 3.416 to 58.358°. |
| Index ranges | −14 <= h <= 14, −6 <= k <= |
| | 6, −25 <= l <= 26 |
| Reflections collected | 22149 |
| Independent reflections | 5405 [R(int) = 0.0849] |
| Completeness to theta = | 96.9% |
| 58.358° | |
| Absorption correction | Empirical |
| Refinement method | Full-matrix least-squares on F² |
| Data/restraints/parameters | 5405/9/476 |
| Goodness-of-fit on F² | 1.074 |
| Final R indices [I > 2sigma(I)] | R1 = 0.0659, wR2 = 0.1680 |
| R indices (all data) | R1 = 0.0821, wR2 = 0.1786 |
| Absolute structure parameter | 0.12(6) |
| Extinction coefficient | n/a |
| Largest diff. peak and hole | 0.301 and −0.346 e · Å⁻³ |

TABLE E2-2
Atomic coordinates (×10⁴) and equivalent isotropic displacement
parameters (Å² × 10³) for Form 2. U(eq) is defined as one
third of the trace of the orthogonalized U^{ij} tensor.

| | x | | y | | z | | U(eq) | |
|---|---|---|---|---|---|---|---|---|
| Cl(1) | 2581 | (4) | 11780 | (30) | 6569 | (4) | 378 | (7) |
| N(1) | 5044 | (11) | 8410 | (30) | 6117 | (5) | 175 | (5) |
| C(1) | 4152 | (16) | 9050 | (60) | 6370 | (8) | 225 | (12) |
| C(2) | 3718 | (14) | 10890 | (60) | 6277 | (12) | 219 | (13) |
| C(3) | 4300 | (30) | 12470 | (60) | 5989 | (14) | 286 | (17) |
| C(4) | 5186 | (17) | 11870 | (40) | 5789 | (11) | 227 | (10) |
| C(5) | 5581 | (10) | 9900 | (20) | 5840 | (5) | 126 | (4) |
| N(2) | 9326 | (3) | 11743 | (8) | 7589 | (2) | 54 | (1) |
| N(3) | 10176 | (3) | 8565 | (8) | 8754 | (2) | 52 | (1) |
| N(4) | 8507 | (3) | 8823 | (7) | 8496 | (2) | 47 | (1) |
| N(5) | 3667 | (3) | 3409 | (8) | 9569 | (2) | 49 | (1) |
| O(1) | 6700 | (8) | 10298 | (17) | 5094 | (3) | 151 | (3) |
| O(2) | 7372 | (5) | 10295 | (14) | 5994 | (2) | 122 | (2) |
| O(3) | 6798 | (5) | 6442 | (10) | 7818 | (2) | 103 | (2) |
| O(4) | 6517 | (3) | 2465 | (7) | 9413 | (2) | 62 | (1) |
| O(5) | 7791 | (3) | 641 | (7) | 9848 | (2) | 64 | (1) |
| O(6) | 3401 | (3) | 5679 | (7) | 8537 | (2) | 75 | (1) |
| O(7) | 4901 | (3) | 86 | (6) | 9094 | (2) | 62 | (1) |
| O(8) | 2255 | (3) | −731 | (6) | 8733 | (2) | 61 | (1) |
| C(6) | 6593 | (9) | 9250 | (20) | 5626 | (4) | 118 | (3) |
| C(7) | 6806 | (13) | 6880 | (20) | 5590 | (7) | 167 | (5) |
| C(8) | 8023 | (8) | 11410 | (16) | 5654 | (3) | 98 | (3) |
| C(9) | 7637 | (10) | 11460 | (20) | 5116 | (3) | 116 | (3) |
| C(10) | 8078 | (12) | 12520 | (30) | 4692 | (4) | 142 | (4) |
| C(11) | 8984 | (13) | 13550 | (30) | 4828 | (4) | 155 | (5) |
| C(12) | 9410 | (9) | 13510 | (20) | 5362 | (3) | 128 | (4) |
| C(13) | 8937 | (7) | 12450 | (16) | 5800 | (3) | 96 | (3) |
| C(14) | 9378 | (6) | 12511 | (14) | 6388 | (3) | 83 | (2) |
| C(15) | 8732 | (6) | 13879 | (13) | 6775 | (3) | 82 | (2) |
| C(16) | 9212 | (6) | 13926 | (11) | 7362 | (3) | 75 | (2) |
| C(17) | 9980 | (5) | 10417 | (12) | 7242 | (2) | 71 | (2) |
| C(18) | 9549 | (6) | 10275 | (14) | 6651 | (3) | 83 | (2) |
| C(19) | 9680 | (4) | 11785 | (10) | 8170 | (2) | 58 | (1) |
| C(20) | 9472 | (4) | 9706 | (9) | 8468 | (2) | 48 | (1) |
| C(21) | 7527 | (4) | 9701 | (10) | 8270 | (2) | 55 | (1) |
| C(22) | 7151 | (4) | 8630 | (12) | 7742 | (2) | 65 | (2) |
| C(23) | 6088 | (7) | 9320 | (20) | 7521 | (4) | 112 | (3) |
| C(24) | 5793 | (8) | 7010 | (30) | 7585 | (5) | 145 | (5) |
| C(25) | 8600 | (4) | 6993 | (9) | 8828 | (2) | 46 | (1) |
| C(26) | 9649 | (4) | 6850 | (9) | 8990 | (2) | 46 | (1) |
| C(27) | 9995 | (4) | 5194 | (10) | 9341 | (2) | 50 | (1) |
| C(28) | 9282 | (4) | 3703 | (10) | 9519 | (2) | 48 | (1) |
| C(29) | 8227 | (4) | 3843 | (9) | 9353 | (2) | 45 | (1) |
| C(30) | 7881 | (4) | 5488 | (9) | 8998 | (2) | 45 | (1) |
| C(31) | 7460 | (4) | 2181 | (9) | 9559 | (2) | 46 | (1) |
| C(32) | 3324 | (4) | 2306 | (9) | 9042 | (2) | 47 | (1) |
| C(33) | 2752 | (4) | 3898 | (9) | 8662 | (2) | 57 | (1) |
| C(34) | 4280 | (4) | 1420 | (9) | 8750 | (2) | 52 | (1) |
| C(35) | 2607 | (4) | 435 | (10) | 9209 | (2) | 54 | (1) |
| O(1W) | 5386 | (3) | 6086 | (7) | 9518 | (2) | 62 | (1) |

TABLE E2-3
Bond lengths [Å] and angles [°] for Form 2.

| Cl(1)—C(2) | 1.75 | (2) | N(2)—C(16) | 1.469 | (9) |
|---|---|---|---|---|---|
| N(1)—C(5) | 1.356 | (17) | N(2)—C(17) | 1.473 | (8) |
| N(1)—C(1) | 1.39 | (3) | N(2)—C(19) | 1.498 | (7) |
| C(1)—C(2) | 1.29 | (4) | N(3)—C(20) | 1.340 | (7) |
| C(1)—H(1) | 0.9300 | | N(3)—C(26) | 1.399 | (7) |
| C(2)—C(3) | 1.43 | (4) | N(4)—C(20) | 1.368 | (6) |
| C(3)—C(4) | 1.32 | (3) | N(4)—C(25) | 1.403 | (7) |
| C(3)—H(3) | 0.9300 | | N(4)—C(21) | 1.475 | (7) |
| C(4)—C(5) | 1.33 | (2) | N(5)—C(32) | 1.528 | (7) |
| C(4)—H(4) | 0.9300 | | N(5)—H(5X) | 0.97 | (3) |
| C(5)—C(6) | 1.483 | (18) | N(5)—H(5Y) | 0.98 | (3) |
| N(5)—H(5Z) | 0.99 | (3) | C(16)—H(16B) | 0.9700 | |
| O(1)—C(9) | 1.410 | (14) | C(17)—C(18) | 1.554 | (10) |
| O(1)—C(6) | 1.479 | (13) | C(17)—H(17A) | 0.9700 | |
| O(2)—C(8) | 1.392 | (10) | C(17)—H(17B) | 0.9700 | |
| O(2)—C(6) | 1.486 | (12) | C(18)—H(18A) | 0.9700 | |
| O(3)—C(22) | 1.445 | (9) | C(18)—H(18B) | 0.9700 | |
| O(3)—C(24) | 1.450 | (12) | C(19)—C(20) | 1.512 | (8) |
| O(4)—C(31) | 1.274 | (6) | C(19)—H(19A) | 0.9700 | |
| O(5)—C(31) | 1.261 | (7) | C(19)—H(19B) | 0.9700 | |
| O(6)—C(33) | 1.426 | (7) | C(21)—C(22) | 1.534 | (9) |
| O(6)—H(6Z) | 0.99 | (3) | C(21)—H(21A) | 0.9700 | |
| O(7)—C(34) | 1.420 | (7) | C(21)—H(21B) | 0.9700 | |
| O(7)—H(7Z) | 0.99 | (3) | C(22)—C(23) | 1.527 | (10) |
| O(8)—C(35) | 1.445 | (7) | C(22)—H(22) | 0.9800 | |
| O(8)—H(8Z) | 0.97 | (3) | C(23)—C(24) | 1.493 | (19) |
| C(6)—C(7) | 1.50 | (2) | C(23)—H(23A) | 0.9700 | |
| C(7)—H(7A) | 0.9600 | | C(23)—H(23B) | 0.9700 | |
| C(7)—H(7B) | 0.9600 | | C(24)—H(24A) | 0.9700 | |
| C(7)—H(7C) | 0.9600 | | C(24)—H(24B) | 0.9700 | |
| C(8)—C(13) | 1.386 | (13) | C(25)—C(30) | 1.391 | (7) |
| C(8)—C(9) | 1.410 | (12) | C(25)—C(26) | 1.406 | (7) |
| C(9)—C(10) | 1.376 | (16) | C(26)—C(27) | 1.410 | (8) |
| C(10)—C(11) | 1.371 | (18) | C(27)—C(28) | 1.386 | (8) |
| C(10)—H(10) | 0.9300 | | C(27)—H(27) | 0.9300 | |
| C(11)—C(12) | 1.418 | (16) | C(28)—C(29) | 1.416 | (7) |
| C(11)—H(11) | 0.9300 | | C(28)—H(28) | 0.9300 | |
| C(12)—C(13) | 1.422 | (12) | C(29)—C(30) | 1.411 | (8) |
| C(12)—H(12) | 0.9300 | | C(29)—C(31) | 1.528 | (7) |
| C(13)—C(14) | 1.548 | (11) | C(30)—H(30) | 0.9300 | |
| C(14)—C(18) | 1.544 | (11) | C(32)—C(33) | 1.538 | (8) |
| C(14)—C(15) | 1.545 | (10) | C(32)—C(35) | 1.549 | (8) |
| C(14)—H(14) | 0.9800 | | C(32)—C(34) | 1.552 | (7) |
| C(15)—C(16) | 1.565 | (10) | C(33)—H(33A) | 0.9700 | |
| C(15)—H(15A) | 0.9700 | | C(33)—H(33B) | 0.9700 | |
| C(15)—H(15B) | 0.9700 | | C(34)—H(34A) | 0.9700 | |
| C(16)—H(16A) | 0.9700 | | C(34)—H(34B) | 0.9700 | |
| C(35)—H(35A) | 0.9700 | | C(22)—O(3)—C(24) | 90.2 | (8) |
| C(35)—H(35B) | 0.9700 | | C(33)—O(6)—H(6Z) | 110 | (5) |
| O(1W)—H(1WX) | 0.99 | (3) | C(34)—O(7)—H(7Z) | 110 | (4) |
| O(1W)—H(1WY) | 1.00 | (3) | C(35)—O(8)—H(8Z) | 113 | (4) |
| | | | O(1)—C(6)—C(5) | 107.9 | (9) |
| C(5)—N(1)—C(1) | 119 | (2) | O(1)—C(6)—O(2) | 106.1 | (9) |
| C(2)—C(1)—N(1) | 122 | (2) | C(5)—C(6)—O(2) | 104.7 | (9) |
| C(2)—C(1)—H(1) | 118.8 | | O(1)—C(6)—C(7) | 110.7 | (11) |
| N(1)—C(1)—H(1) | 118.8 | | C(5)—C(6)—C(7) | 116.9 | (13) |
| C(1)—C(2)—C(3) | 117 | (2) | O(2)—C(6)—C(7) | 109.8 | (10) |
| C(1)—C(2)—Cl(1) | 125 | (3) | C(6)—C(7)—H(7A) | 109.5 | |
| C(3)—C(2)—Cl(1) | 117 | (3) | C(6)—C(7)—H(7B) | 109.5 | |
| C(4)—C(3)—C(2) | 118 | (3) | H(7A)—C(7)—H(7B) | 109.5 | |
| C(4)—C(3)—H(3) | 120.8 | | C(6)—C(7)—H(7C) | 109.5 | |
| C(2)—C(3)—H(3) | 120.8 | | H(7A)—C(7)—H(7C) | 109.5 | |
| C(3)—C(4)—C(5) | 124 | (3) | H(7B)—C(7)—H(7C) | 109.5 | |
| C(3)—C(4)—H(4) | 118.0 | | C(13)—C(8)—O(2) | 126.8 | (7) |
| C(5)—C(4)—H(4) | 118.0 | | C(13)—C(8)—C(9) | 120.9 | (8) |
| C(4)—C(5)—N(1) | 118.2 | (17) | O(2)—C(8)—C(9) | 112.3 | (9) |
| C(4)—C(5)—C(6) | 123.8 | (15) | C(10)—C(9)—C(8) | 126.1 | (12) |
| N(1)—C(5)—C(6) | 118.0 | (13) | C(10)—C(9)—O(1) | 126.1 | (10) |
| C(16)—N(2)—C(17) | 110.1 | (5) | C(8)—C(9)—O(1) | 107.8 | (8) |
| C(16)—N(2)—C(19) | 112.0 | (5) | C(11)—C(10)—C(9) | 113.9 | (10) |
| C(17)—N(2)—C(19) | 113.9 | (5) | C(11)—C(10)—H(10) | 123.0 | |
| C(20)—N(3)—C(26) | 106.8 | (4) | C(9)—C(10)—H(10) | 123.0 | |
| C(20)—N(4)—C(25) | 106.9 | (4) | C(10)—C(11)—C(12) | 121.8 | (11) |
| C(20)—N(4)—C(21) | 127.5 | (5) | C(10)—C(11)—H(11) | 119.1 | |
| C(25)—N(4)—C(21) | 125.3 | (4) | C(12)—C(11)—H(11) | 119.1 | |
| C(32)—N(5)—H(5X) | 118 | (3) | C(11)—C(12)—C(13) | 123.9 | (12) |
| C(32)—N(5)—H(5Y) | 106 | (3) | C(11)—C(12)—H(12) | 118.1 | |
| H(5X)—N(5)—H(5Y) | 107 | (5) | C(13)—C(12)—H(12) | 118.1 | |
| C(32)—N(5)—H(5Z) | 117 | (4) | C(8)—C(13)—C(12) | 113.4 | (8) |
| H(5X)—N(5)—H(5Z) | 90 | (5) | C(8)—C(13)—C(14) | 123.1 | (6) |
| H(5Y)—N(5)—H(5Z) | 118 | (5) | C(12)—C(13)—C(14) | 123.5 | (9) |
| C(9)—O(1)—C(6) | 107.0 | (7) | C(18)—C(14)—C(15) | 107.6 | (5) |
| C(8)—O(2)—C(6) | 104.8 | (7) | C(18)—C(14)—C(13) | 114.8 | (7) |
| C(15)—C(14)—C(13) | 114.0 | (7) | N(4)—C(20)—C(19) | 122.8 | (5) |
| C(18)—C(14)—H(14) | 106.6 | | N(4)—C(21)—C(22) | 114.3 | (5) |
| C(15)—C(14)—H(14) | 106.6 | | N(4)—C(21)—H(21A) | 108.7 | |
| C(13)—C(14)—H(14) | 106.6 | | C(22)—C(21)—H(21A) | 108.7 | |
| C(14)—C(15)—C(16) | 112.4 | (6) | N(4)—C(21)—H(21B) | 108.7 | |
| C(14)—C(15)—H(15A) | 109.1 | | C(22)—C(21)—H(21B) | 108.7 | |
| C(16)—C(15)—H(15A) | 109.1 | | H(21A)—C(21)—H(21B) | 107.6 | |
| C(14)—C(15)—H(15B) | 109.1 | | O(3)—C(22)—C(23) | 91.5 | (6) |
| C(16)—C(15)—H(15B) | 109.1 | | O(3)—C(22)—C(21) | 112.8 | (5) |
| H(15A)—C(15)—H(15B) | 107.9 | | C(23)—C(22)—C(21) | 116.3 | (7) |
| N(2)—C(16)—C(15) | 111.7 | (6) | O(3)—C(22)—H(22) | 111.6 | |
| N(2)—C(16)—H(16A) | 109.3 | | C(23)—C(22)—H(22) | 111.6 | |
| C(15)—C(16)—H(16A) | 109.3 | | C(21)—C(22)—H(22) | 111.6 | |
| N(2)—C(16)—H(16B) | 109.3 | | C(24)—C(23)—C(22) | 85.5 | (8) |
| C(15)—C(16)—H(16B) | 109.3 | | C(24)—C(23)—H(23A) | 114.4 | |
| H(16A)—C(16)—H(16B) | 107.9 | | C(22)—C(23)—H(23A) | 114.4 | |
| N(2)—C(17)—C(18) | 112.6 | (5) | C(24)—C(23)—H(23B) | 114.4 | |
| N(2)—C(17)—H(17A) | 109.1 | | C(22)—C(23)—H(23B) | 114.4 | |
| C(18)—C(17)—H(17A) | 109.1 | | H(23A)—C(23)—H(23B) | 111.5 | |
| N(2)—C(17)—H(17B) | 109.1 | | O(3)—C(24)—C(23) | 92.7 | (8) |
| C(18)—C(17)—H(17B) | 109.1 | | O(3)—C(24)—H(24A) | 113.2 | |
| H(17A)—C(17)—H(17B) | 107.8 | | C(23)—C(24)—H(24A) | 113.2 | |
| C(14)—C(18)—C(17) | 113.0 | (6) | O(3)—C(24)—H(24B) | 113.2 | |
| C(14)—C(18)—H(18A) | 109.0 | | C(23)—C(24)—H(24B) | 113.2 | |
| C(17)—C(18)—H(18A) | 109.0 | | H(24A)—C(24)—H(24B) | 110.5 | |
| C(14)—C(18)—H(18B) | 109.0 | | C(30)—C(25)—N(4) | 132.2 | (4) |
| C(17)—C(18)—H(18B) | 109.0 | | C(30)—C(25)—C(26) | 121.4 | (5) |
| H(18A)—C(18)—H(18B) | 107.8 | | N(4)—C(25)—C(26) | 106.4 | (4) |
| N(2)—C(19)—C(20) | 113.6 | (5) | N(3)—C(26)—C(25) | 108.2 | (5) |
| N(2)—C(19)—H(19A) | 108.8 | | N(3)—C(26)—C(27) | 131.3 | (4) |
| C(20)—C(19)—H(19A) | 108.8 | | C(25)—C(26)—C(27) | 120.5 | (5) |
| N(2)—C(19)—H(19B) | 108.8 | | C(28)—C(27)—C(26) | 118.6 | (4) |
| C(20)—C(19)—H(19B) | 108.8 | | C(28)—C(27)—H(27) | 120.7 | |
| H(19A)—C(19)—H(19B) | 107.7 | | C(26)—C(27)—H(27) | 120.7 | |
| N(3)—C(20)—N(4) | 111.7 | (5) | C(27)—C(28)—C(29) | 120.8 | (5) |

| N(3)—C(20)—C(19) | 125.4 | (5) | C(27)—C(28)—H(28) | 119.6 |
|---|---|---|---|---|
| C(29)—C(28)—H(28) | 119.6 | | | |
| C(30)—C(29)—C(28) | 120.7 | (5) | | |
| C(30)—C(29)—C(31) | 119.8 | (4) | | |
| C(28)—C(29)—C(31) | 119.5 | (5) | | |
| C(25)—C(30)—C(29) | 118.0 | (4) | | |
| C(25)—C(30)—H(30) | 121.0 | | | |
| C(29)—C(30)—H(30) | 121.0 | | | |
| O(5)—C(31)—O(4) | 124.9 | (5) | | |
| O(5)—C(31)—C(29) | 119.1 | (4) | | |
| O(4)—C(31)—C(29) | 116.0 | (5) | | |
| N(5)—C(32)—C(33) | 111.0 | (4) | | |
| N(5)—C(32)—C(35) | 105.5 | (4) | | |
| C(33)—C(32)—C(35) | 111.2 | (4) | | |
| N(5)—C(32)—C(34) | 110.0 | (4) | | |
| C(33)—C(32)—C(34) | 108.5 | (4) | | |
| C(35)—C(32)—C(34) | 110.7 | (4) | | |
| O(6)—C(33)—C(32) | 110.6 | (4) | | |
| O(6)—C(33)—H(33A) | 109.5 | | | |
| C(32)—C(33)—H(33A) | 109.5 | | | |
| O(6)—C(33)—H(33B) | 109.5 | | | |
| C(32)—C(33)—H(33B) | 109.5 | | | |
| H(33A)—C(33)—H(33B) | 108.1 | | | |
| O(7)—C(34)—C(32) | 111.7 | (4) | | |
| O(7)—C(34)—H(34A) | 109.3 | | | |
| C(32)—C(34)—H(34A) | 109.3 | | | |
| O(7)—C(34)—H(34B) | 109.3 | | | |
| C(32)—C(34)—H(34B) | 109.3 | | | |
| H(34A)—C(34)—H(34B) | 107.9 | | | |
| O(8)—C(35)—C(32) | 109.4 | (4) | | |
| O(8)—C(35)—H(35A) | 109.8 | | | |
| C(32)—C(35)—H(35A) | 109.8 | | | |
| O(8)—C(35)—H(35B) | 109.8 | | | |
| C(32)—C(35)—H(35B) | 109.8 | | | |
| H(35A)—C(35)—H(35B) | 108.2 | | | |
| H(1WX)—O(1W)—H(1WY) | 104 | (6) | | |

Symmetry transformations used to generate equivalent atoms:

TABLE E2-4
Anisotropic displacement parameters (Å² × 10³) for Form 2.
The anisotropic displacement factor exponent takes the
form: −2π²[h² a*²U¹¹ + . . . + 2 h k a* b* U¹²]

| | U¹¹ | U²² | U³³ | U²³ | U¹³ | U¹² |
|---|---|---|---|---|---|---|
| Cl(1) | 135(3) | 610(20) | 393(10) | −72(14) | 34(5) | 33(7) |
| N(1) | 154(10) | 208(13) | 161(9) | 50(10) | −32(8) | −41(10) |
| C(1) | 108(12) | 390(40) | 175(15) | 30(20) | −3(11) | −59(18) |
| C(2) | 99(11) | 320(40) | 230(20) | −30(20) | −48(12) | 48(16) |
| C(3) | 180(20) | 280(40) | 390(40) | −50(30) | 0(30) | 60(30) |
| C(4) | 167(16) | 169(18) | 350(30) | 60(20) | 34(17) | 21(14) |
| C(5) | 127(9) | 134(10) | 113(7) | 39(7) | −32(6) | −16(8) |
| N(2) | 60(3) | 46(3) | 56(2) | 3(2) | 10(2) | −2(2) |
| N(3) | 46(2) | 56(3) | 55(2) | −4(2) | 2(2) | −13(2) |
| N(4) | 40(2) | 52(3) | 51(2) | 1(2) | 2(2) | −3(2) |
| N(5) | 43(2) | 46(3) | 58(3) | −5(2) | 2(2) | 2(2) |
| O(1) | 184(8) | 173(8) | 92(4) | 21(5) | −40(5) | −9(7) |
| O(2) | 115(4) | 173(7) | 77(3) | 35(4) | −21(3) | −36(5) |
| O(3) | 103(4) | 95(4) | 108(4) | −3(3) | −39(3) | −16(3) |
| O(4) | 38(2) | 60(3) | 87(3) | 6(2) | −1(2) | −8(2) |
| O(5) | 48(2) | 64(3) | 79(3) | 19(2) | 3(2) | −6(2) |
| O(6) | 69(3) | 48(2) | 109(3) | 14(2) | 17(2) | 3(2) |
| O(7) | 44(2) | 48(2) | 92(3) | −2(2) | −3(2) | 4(2) |
| O(8) | 42(2) | 52(2) | 90(3) | −11(2) | 1(2) | −7(2) |
| C(6) | 127(8) | 135(10) | 91(6) | 3(6) | −21(6) | −10(7) |
| C(7) | 185(13) | 125(11) | 189(13) | 22(10) | −43(10) | 0(10) |
| C(8) | 122(7) | 109(7) | 63(4) | 27(4) | 13(4) | 2(6) |
| C(9) | 151(9) | 126(8) | 70(5) | 15(5) | −16(5) | 3(7) |
| C(10) | 184(12) | 177(12) | 65(5) | 24(6) | 1(6) | 4(10) |
| C(11) | 203(13) | 186(13) | 79(6) | 45(7) | 33(7) | −10(12) |
| C(12) | 157(9) | 158(10) | 70(5) | 32(6) | 16(5) | −10(8) |
| C(13) | 117(7) | 106(6) | 67(4) | 14(4) | 24(4) | −3(6) |
| C(14) | 97(5) | 92(5) | 61(4) | 19(4) | 13(3) | −7(4) |
| C(15) | 106(5) | 58(4) | 82(4) | 8(4) | 5(4) | 13(4) |
| C(16) | 92(5) | 53(4) | 79(4) | 0(3) | 6(4) | 6(4) |
| C(17) | 77(4) | 71(4) | 67(4) | 10(3) | 16(3) | 17(4) |
| C(18) | 102(5) | 84(5) | 64(4) | −4(4) | 27(3) | 23(4) |
| C(19) | 57(3) | 55(4) | 61(3) | 3(3) | 2(3) | −9(3) |
| C(20) | 39(3) | 55(3) | 48(3) | −7(2) | 1(2) | −6(2) |
| C(21) | 45(3) | 58(4) | 61(3) | 2(3) | 2(2) | 4(3) |
| C(22) | 50(3) | 82(5) | 61(3) | 5(3) | −2(3) | −1(3) |
| C(23) | 76(5) | 148(10) | 110(6) | 14(6) | −29(5) | 6(6) |
| C(24) | 84(6) | 196(14) | 152(9) | 15(10) | −36(6) | −32(8) |
| C(25) | 40(3) | 55(3) | 43(2) | −3(2) | 2(2) | 0(2) |
| C(26) | 37(2) | 53(3) | 46(2) | −7(3) | 2(2) | −3(2) |
| C(27) | 34(2) | 63(4) | 52(3) | −5(3) | −3(2) | −3(2) |
| C(28) | 44(3) | 57(3) | 44(2) | 1(3) | −4(2) | −4(3) |
| C(29) | 42(3) | 53(3) | 41(2) | −3(2) | 1(2) | −4(2) |
| C(30) | 36(2) | 53(3) | 47(2) | 0(2) | 4(2) | −5(2) |
| C(31) | 45(3) | 48(3) | 46(3) | −1(3) | 3(2) | −1(2) |
| C(32) | 36(2) | 47(3) | 57(3) | 0(2) | 1(2) | −1(2) |
| C(33) | 48(3) | 47(3) | 76(3) | 1(3) | 3(3) | 4(3) |
| C(34) | 41(3) | 50(3) | 66(3) | −5(3) | 4(2) | −2(2) |
| C(35) | 42(3) | 53(3) | 67(3) | −3(3) | 5(2) | −4(3) |
| O(1W) | 52(2) | 54(2) | 80(3) | 4(2) | 5(2) | −2(2) |

### Calculated/Simulated PXRD Data.

Using the information obtained by Single Crystal X-Ray Analysis herein above, PXRD peak positions and intensity for Form 2 can be calculated/simulated (See FIG. 4, using Bruker DIFFRAC.EVA version 5.0.0.22). A list of calculated/simulated PXRD diffraction peaks expressed in terms of the degree 2θ and relative intensities with a relative intensity of 3.0% for Form 2 is provided below.

TABLE E2-5
Calculated PXRD peak positions and intensity for Form 2.

| Angle 2-Theta ° | Relative Intensity % |
|---|---|
| 3.6 | 100% |
| 7.1 | 96% |
| 7.6 | 69% |
| 7.8 | 37% |
| 9.7 | 22% |
| 10.0 | 9% |
| 10.7 | 11% |
| 12.5 | 5% |
| 14.0 | 18% |
| 14.3 | 37% |
| 14.7 | 42% |
| 15.6 | 18% |
| 16.0 | 31% |
| 16.2 | 25% |
| 17.3 | 57% |
| 17.9 | 15% |
| 19.0 | 19% |
| 19.4 | 57% |
| 19.8 | 42% |
| 20.2 | 23% |
| 20.6 | 7% |
| 20.8 | 10% |
| 21.0 | 7% |
| 21.3 | 14% |
| 21.5 | 6% |
| 22.4 | 21% |
| 22.9 | 11% |
| 23.8 | 24% |
| 24.3 | 23% |
| 24.8 | 4% |
| 25.9 | 9% |
| 26.4 | 5% |
| 26.6 | 11% |
| 27.0 | 10% |
| 28.7 | 5% |
| 29.1 | 6% |
| 29.7 | 4% |
| 30.0 | 8% |
| 31.5 | 7% |
| 32.3 | 3% |
| 34.1 | 3% |
| 35.8 | 4% |
| 36.2 | 4% |

### Example 3. Form 3 of Tris Salt of Compound 1

### Preparation of Form 3 of Tris Salt of Compound 1 (Slurry to Slurry Conversion)

The anhydrous form Form A of tris salt of Compound 1 (1.177 grams) was added to a 50 mL EasyMax® reactor. A mixed solvent of acetonitrile and water (27.9 mL acetonitrile and 2.4 mL water) was then added. The resulting mixture (a slurry) was stirred with overhead paddle stirring at room temperature (about 25° C.) over two days. The mixture was then cooled to 0° C. and stirred for about 1 hour. Then the mixture was filtered by suction filtration through filter paper and the solid collected (cake) was rinsed with 2-3 mL cold acetonitrile (0° C.) twice. The resulting cake was air-dried on the funnel for one hour. The cake/funnel was transferred to a vacuum oven for further drying (50° C./˜22 in Hg vacuum, with slight nitrogen bleed). After about 5 hours 1.115 gm of white solid was obtained (designed as Form 3).

### Alternative Preparation of Form 3 of Compound 1, 1,3-dihydroxy-2-(hydroxymethyl)propan-2-aminium salt

Alternatively, single crystals of Form 3 of tris salt of Compound 1 were prepared by vapor diffusion of acetonitrile into a saturated solution of Compound 1, 1,3-dihydroxy-2-(hydroxymethyl)propan-2-aminium salt in acetonitrile/15% water (v/v).

### Single Crystal X-Ray Analysis.

A sample of Form 3 of tris salt of Compound 1 was tested for single crystal X-ray analysis. Data collection was performed on a Bruker D8 Venture diffractometer at room temperature on a representative crystal. Data collection consisted of omega and phi scans.

The structure was solved by intrinsic phasing using SHELX software suite (SHELXTL, Version 5.1, Bruker AXS, 1997) in the Monoclinic space group P2₁. The structure was subsequently refined by the full-matrix least squares method. All non-hydrogen atoms were found and refined using anisotropic displacement parameters.

The hydrogen atoms located on nitrogen and oxygen were found from the Fourier difference map and refined with distances restrained. The remaining hydrogen atoms were placed in calculated positions and were allowed to ride on their carrier atoms. The final refinement included isotropic displacement parameters for all hydrogen atoms.

Analysis of the absolute structure using likelihood methods (See R. W. W. Hooft et al. J. Appl. Cryst. (2008). 41. 96-103) was performed using PLATON (See A. L. Spek, J. Appl. Cryst. 2003, 36, 7-13). Assuming the sample submitted is enantiopure, the absolute structure (with stereochemistry information on the two chiral centers) was assigned.

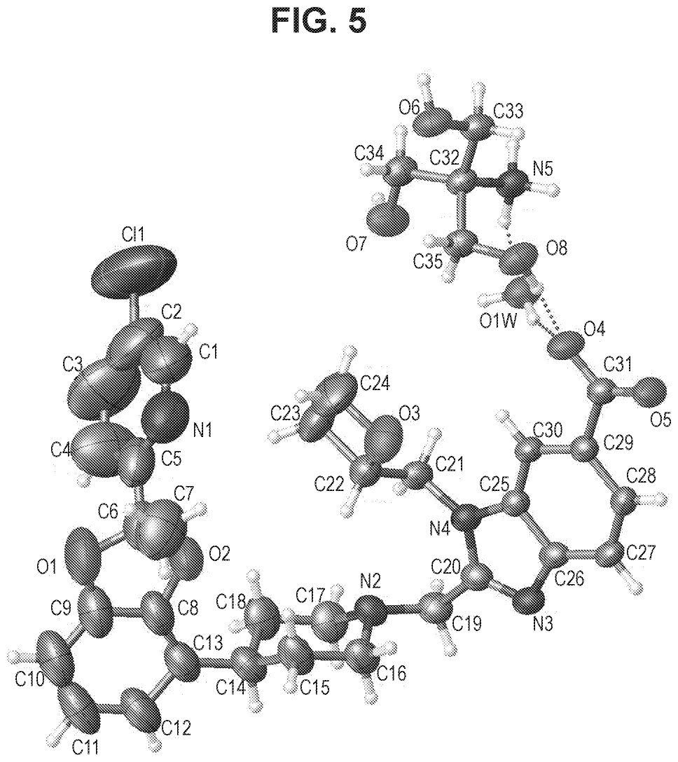

The final R-index was 5.1%. A final difference Fourier revealed no missing or misplaced electron density. The refined structure was plotted using the SHELXTL plotting package (SHELXTL, Version 5.1, Bruker AXS, 1997) (FIG. 5). The absolute configuration was determined by the method of Flack (See H. D. Flack, Acta Cryst. 1983, A39, 867-881). According to the refined structure, Form 3 is a monohydrate of tris salt of Compound 1:

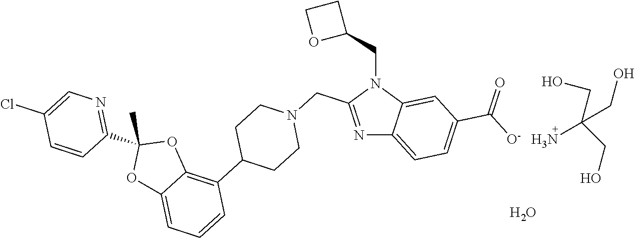 [SMILES: C[C@]1(c2ccc(Cl)cn2)Oc2cccc(C3CCN(Cc4nc5ccc(C(=O)[O-])cc5n4C[C@@H]4CCO4)CC3)c2O1 ; O ; NC(CO)(CO)CO]

and a chemical name for this hydrate form (including stereochemistry information) is: 2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylate, 1,3-dihydroxy-2-(hydroxymethyl)propan-2-aminium salt, monohydrate.

Pertinent crystal, data collection and refinement are summarized in Table E3-1. Atomic coordinates, bond lengths, bond angles and displacement parameters are listed in tables E3-2 to E3-4.

TABLE E3-1
Crystal data and structure refinement for Form 3.

| Empirical formula | C35 H44 Cl N5 O9 | |
|---|---|---|
| Formula weight | 714.20 | |
| Temperature | 296(2) K | |
| Wavelength | 1.54178 Å | |
| Crystal system | Monoclinic | |
| Space group | P2₁ | |
| Unit cell dimensions | a = 12.8892(5) Å | α = 90°. |
| | b = 6.1536(3) Å | β = 91.835(2)°. |
| | c = 23.9167(10) Å | γ = 90°. |
| Volume | 1895.98(14) Å³ | |

| | |
|---|---|
| Z | 2 |
| Density (calculated) | 1.251 Mg/m³ |
| Absorption coefficient | 1.373 mm⁻¹ |
| F(000) | 756 |
| Crystal size | 0.780 × 0.100 × 0.040 mm³ |
| Theta range for data collection | 3.431 to 72.528°. |
| Index ranges | −12 <= h <= 15, −7 <= k <= |
| | 7, −29 <= l <= 29 |
| Reflections collected | 16800 |
| Independent reflections | 6869 [R(int) = 0.0523] |
| Completeness to theta = | 98.0% |
| 67.679° | |
| Absorption correction | Empirical |
| Refinement method | Full-matrix least-squares on F² |
| Data/restraints/parameters | 6869/9/476 |
| Goodness-of-fit on F² | 1.043 |
| Final R indices [I > 2sigma(I)] | R1 = 0.0508, wR2 = 0.1434 |
| R indices (all data) | R1 = 0.0542, wR2 = 0.1482 |
| Absolute structure parameter | 0.06(3) |
| Extinction coefficient | n/a |
| Largest diff. peak and hole | 0.260 and −0.321 e · Å⁻³ |

TABLE E3-2
Atomic coordinates (×10⁴) and equivalent isotropic displacement
parameters (Å² × 10³) for Form 3. U(eq) is defined as one
third of the trace of the orthogonalized U^{ij} tensor.

| | x | | y | | z | | U(eq) | |
|---|---|---|---|---|---|---|---|---|
| Cl(1) | 2582 | (2) | 9965 | (16) | 6603 | (2) | 325 | (4) |
| N(1) | 5060 | (6) | 6766 | (14) | 6129 | (3) | 148 | (2) |
| N(2) | 9316 | (2) | 10129 | (4) | 7585 | (1) | 49 | (1) |
| N(3) | 10186 | (2) | 6949 | (4) | 8750 | (1) | 48 | (1) |
| N(4) | 8505 | (2) | 7208 | (4) | 8496 | (1) | 44 | (1) |
| N(5) | 3669 | (2) | 1786 | (4) | 9568 | (1) | 47 | (1) |
| O(1) | 6678 | (4) | 8716 | (10) | 5096 | (2) | 131 | (2) |
| O(2) | 7358 | (3) | 8701 | (8) | 6001 | (1) | 104 | (1) |
| O(3) | 6794 | (3) | 4812 | (6) | 7825 | (1) | 92 | (1) |
| O(4) | 6522 | (2) | 848 | (4) | 9422 | (1) | 58 | (1) |
| O(5) | 7796 | (2) | −981 | (4) | 9855 | (1) | 60 | (1) |
| O(6) | 2262 | (2) | −2325 | (4) | 8721 | (1) | 56 | (1) |
| O(7) | 3425 | (2) | 4070 | (4) | 8536 | (1) | 69 | (1) |
| O(8) | 4910 | (2) | −1536 | (3) | 9093 | (1) | 58 | (1) |
| O(1W) | 5392 | (2) | 4478 | (4) | 9523 | (1) | 58 | (1) |
| C(1) | 4178 | (9) | 7380 | (30) | 6380 | (4) | 185 | (5) |
| C(2) | 3714 | (8) | 9290 | (40) | 6315 | (6) | 190 | (6) |
| C(3) | 4194 | (12) | 10740 | (30) | 6016 | (7) | 229 | (7) |
| C(4) | 5148 | (9) | 10270 | (20) | 5778 | (6) | 186 | (4) |
| C(5) | 5568 | (5) | 8290 | (13) | 5846 | (2) | 113 | (2) |
| C(6) | 6587 | (5) | 7667 | (12) | 5634 | (2) | 107 | (2) |
| C(7) | 6794 | (7) | 5276 | (14) | 5590 | (4) | 150 | (3) |
| C(8) | 8020 | (4) | 9821 | (9) | 5659 | (2) | 87 | (1) |
| C(9) | 7605 | (5) | 9845 | (11) | 5117 | (2) | 104 | (2) |
| C(10) | 8072 | (7) | 10888 | (14) | 4692 | (2) | 126 | (2) |
| C(11) | 8964 | (7) | 11915 | (15) | 4824 | (2) | 131 | (2) |
| C(12) | 9397 | (5) | 11903 | (12) | 5359 | (2) | 107 | (2) |
| C(13) | 8917 | (4) | 10851 | (8) | 5804 | (2) | 81 | (1) |
| C(14) | 9376 | (3) | 10911 | (7) | 6388 | (2) | 73 | (1) |
| C(15) | 9545 | (3) | 8656 | (7) | 6648 | (1) | 73 | (1) |
| C(16) | 9983 | (3) | 8811 | (6) | 7242 | (1) | 64 | (1) |
| C(17) | 9203 | (3) | 12329 | (6) | 7363 | (2) | 68 | (1) |
| C(18) | 8725 | (4) | 12292 | (6) | 6778 | (2) | 76 | (1) |
| C(19) | 9678 | (2) | 10173 | (5) | 8169 | (1) | 54 | (1) |
| C(20) | 9475 | (2) | 8082 | (5) | 8466 | (1) | 46 | (1) |
| C(21) | 7529 | (2) | 8089 | (5) | 8276 | (1) | 49 | (1) |
| C(22) | 7147 | (3) | 7018 | (7) | 7745 | (1) | 61 | (1) |
| C(23) | 6068 | (3) | 7712 | (11) | 7537 | (2) | 94 | (2) |
| C(24) | 5782 | (5) | 5437 | (16) | 7594 | (3) | 128 | (2) |
| C(25) | 8603 | (2) | 5376 | (5) | 8831 | (1) | 42 | (1) |
| C(26) | 9655 | (2) | 5244 | (5) | 8988 | (1) | 43 | (1) |
| C(27) | 10003 | (2) | 3558 | (5) | 9340 | (1) | 46 | (1) |
| C(28) | 9287 | (2) | 2076 | (5) | 9519 | (1) | 46 | (1) |
| C(29) | 8228 | (2) | 2223 | (5) | 9355 | (1) | 42 | (1) |
| C(30) | 7882 | (2) | 3870 | (5) | 9003 | (1) | 42 | (1) |
| C(31) | 7462 | (2) | 548 | (5) | 9563 | (1) | 45 | (1) |
| C(32) | 3329 | (2) | 681 | (4) | 9035 | (1) | 43 | (1) |
| C(33) | 2609 | (2) | −1171 | (5) | 9201 | (1) | 51 | (1) |
| C(34) | 2769 | (2) | 2309 | (5) | 8659 | (1) | 54 | (1) |
| C(35) | 4294 | (2) | −179 | (5) | 8747 | (1) | 49 | (1) |

TABLE E3-3
Bond lengths [Å] and angles [°] for Form 3.

| Cl(1)—C(2) | 1.684 | (13) | N(5)—C(32) | 1.496 | (4) |
|---|---|---|---|---|---|
| N(1)—C(5) | 1.340 | (10) | N(5)—H(5X) | 0.95 | (2) |
| N(1)—C(1) | 1.355 | (16) | N(5)—H(5Y) | 0.98 | (2) |
| N(2)—C(16) | 1.454 | (4) | N(5)—H(5Z) | 0.97 | (2) |
| N(2)—C(19) | 1.458 | (4) | O(1)—C(9) | 1.382 | (8) |
| N(2)—C(17) | 1.460 | (4) | O(1)—C(6) | 1.446 | (7) |
| N(3)—C(20) | 1.322 | (4) | O(2)—C(8) | 1.385 | (6) |
| N(3)—C(26) | 1.385 | (4) | O(2)—C(6) | 1.452 | (7) |
| N(4)—C(20) | 1.365 | (4) | O(3)—C(22) | 1.446 | (5) |
| N(4)—C(25) | 1.386 | (4) | O(3)—C(24) | 1.451 | (7) |
| N(4)—C(21) | 1.454 | (4) | O(4)—C(31) | 1.261 | (3) |
| O(5)—C(31) | 1.241 | (4) | C(15)—H(15B) | 0.9700 | |
| O(6)—C(33) | 1.410 | (4) | C(16)—H(16A) | 0.9700 | |
| O(6)—H(6Y) | 0.95 | (2) | C(16)—H(16B) | 0.9700 | |
| O(7)—C(34) | 1.411 | (4) | C(17)—C(18) | 1.510 | (5) |
| O(7)—H(7Y) | 0.96 | (3) | C(17)—H(17A) | 0.9700 | |
| O(8)—C(35) | 1.405 | (4) | C(17)—H(17B) | 0.9700 | |
| O(8)—H(8Y) | 0.97 | (2) | C(18)—H(18A) | 0.9700 | |
| O(1W)—H(1WX) | 0.97 | (2) | C(18)—H(18B) | 0.9700 | |
| O(1W)—H(1WY) | 0.96 | (2) | C(19)—C(20) | 1.496 | (4) |
| C(1)—C(2) | 1.33 | (2) | C(19)—H(19A) | 0.9700 | |
| C(1)—H(1) | 0.9300 | | C(19)—H(19B) | 0.9700 | |
| C(2)—C(3) | 1.31 | (2) | C(21)—C(22) | 1.500 | (4) |
| C(3)—C(4) | 1.401 | (17) | C(21)—H(21A) | 0.9700 | |
| C(3)—H(3) | 0.9300 | | C(21)—H(21B) | 0.9700 | |
| C(4)—C(5) | 1.343 | (14) | C(22)—C(23) | 1.522 | (5) |
| C(4)—H(4) | 0.9300 | | C(22)—H(22) | 0.9800 | |
| C(5)—C(6) | 1.473 | (10) | C(23)—C(24) | 1.455 | (11) |
| C(6)—C(7) | 1.500 | (12) | C(23)—H(23A) | 0.9700 | |
| C(7)—H(7A) | 0.9600 | | C(23)—H(23B) | 0.9700 | |
| C(7)—H(7B) | 0.9600 | | C(24)—H(24A) | 0.9700 | |
| C(7)—H(7C) | 0.9600 | | C(24)—H(24B) | 0.9700 | |
| C(8)—C(13) | 1.354 | (7) | C(25)—C(30) | 1.385 | (4) |
| C(8)—C(9) | 1.387 | (6) | C(25)—C(26) | 1.398 | (4) |
| C(9)—C(10) | 1.359 | (9) | C(26)—C(27) | 1.400 | (4) |
| C(10)—C(11) | 1.340 | (11) | C(27)—C(28) | 1.376 | (4) |
| C(10)—H(10) | 0.9300 | | C(27)—H(27) | 0.9300 | |
| C(11)—C(12) | 1.379 | (9) | C(28)—C(29) | 1.411 | (4) |
| C(11)—H(11) | 0.9300 | | C(28)—H(28) | 0.9300 | |
| C(12)—C(13) | 1.405 | (6) | C(29)—C(30) | 1.384 | (4) |
| C(12)—H(12) | 0.9300 | | C(29)—C(31) | 1.521 | (4) |
| C(13)—C(14) | 1.499 | (6) | C(30)—H(30) | 0.9300 | |
| C(14)—C(18) | 1.532 | (6) | C(32)—C(34) | 1.514 | (4) |
| C(14)—C(15) | 1.534 | (6) | C(32)—C(33) | 1.530 | (4) |
| C(14)—H(14) | 0.9800 | | C(32)—C(35) | 1.536 | (4) |
| C(15)—C(16) | 1.514 | (5) | C(33)—H(33A) | 0.9700 | |
| C(15)—H(15A) | 0.9700 | | C(33)—H(33B) | 0.9700 | |
| C(34)—H(34A) | 0.9700 | | C(5)—C(4)—H(4) | 120.3 | |
| C(34)—H(34B) | 0.9700 | | C(3)—C(4)—H(4) | 120.3 | |
| C(35)—H(35A) | 0.9700 | | N(1)—C(5)—C(4) | 119.7 | (9) |
| C(35)—H(35B) | 0.9700 | | N(1)—C(5)—C(6) | 116.6 | (7) |
| | | | C(4)—C(5)—C(6) | 123.7 | (7) |
| C(5)—N(1)—C(1) | 117.3 | (11) | O(1)—C(6)—O(2) | 105.6 | (5) |
| C(16)—N(2)—C(19) | 111.9 | (3) | O(1)—C(6)—C(5) | 106.8 | (5) |
| C(16)—N(2)—C(17) | 111.4 | (3) | O(2)—C(6)—C(5) | 106.1 | (5) |
| C(19)—N(2)—C(17) | 110.9 | (3) | O(1)—C(6)—C(7) | 110.9 | (7) |
| C(20)—N(3)—C(26) | 105.5 | (2) | O(2)—C(6)—C(7) | 110.6 | (6) |
| C(20)—N(4)—C(25) | 106.5 | (2) | C(5)—C(6)—C(7) | 116.3 | (7) |
| C(20)—N(4)—C(21) | 128.1 | (3) | C(6)—C(7)—H(7A) | 109.5 | |
| C(25)—N(4)—C(21) | 125.1 | (2) | C(6)—C(7)—H(7B) | 109.5 | |
| C(32)—N(5)—H(5X) | 105 | (2) | H(7A)—C(7)—H(7B) | 109.5 | |
| C(32)—N(5)—H(5Y) | 112 | (2) | C(6)—C(7)—H(7C) | 109.5 | |
| H(5X)—N(5)—H(5Y) | 104 | (3) | H(7A)—C(7)—H(7C) | 109.5 | |
| C(32)—N(5)—H(5Z) | 111 | (2) | H(7B)—C(7)—H(7C) | 109.5 | |
| H(5X)—N(5)—H(5Z) | 115 | (3) | C(13)—C(8)—O(2) | 128.3 | (4) |
| H(5Y)—N(5)—H(5Z) | 109 | (3) | C(13)—C(8)—C(9) | 122.4 | (4) |
| C(9)—O(1)—C(6) | 106.8 | (4) | O(2)—C(8)—C(9) | 109.3 | (5) |
| C(8)—O(2)—C(6) | 106.5 | (4) | C(10)—C(9)—O(1) | 128.0 | (5) |
| C(22)—O(3)—C(24) | 89.1 | (4) | C(10)—C(9)—C(8) | 122.5 | (7) |
| C(33)—O(6)—H(6Y) | 107 | (2) | O(1)—C(9)—C(8) | 109.6 | (5) |
| C(34)—O(7)—H(7Y) | 105 | (3) | C(11)—C(10)—C(9) | 116.4 | (6) |
| C(35)—O(8)—H(8Y) | 105 | (3) | C(11)—C(10)—H(10) | 121.8 | |
| H(1WX)—O(1W)—H(1WY) | 117 | (3) | C(9)—C(10)—H(10) | 121.8 | |
| C(2)—C(1)—N(1) | 125.3 | (13) | C(10)—C(11)—C(12) | 122.3 | (6) |
| C(2)—C(1)—H(1) | 117.4 | | C(10)—C(11)—H(11) | 118.8 | |
| N(1)—C(1)—H(1) | 117.4 | | C(12)—C(11)—H(11) | 118.8 | |
| C(3)—C(2)—C(1) | 116.5 | (13) | C(11)—C(12)—C(13) | 122.0 | (7) |
| C(3)—C(2)—Cl(1) | 119.2 | (17) | C(11)—C(12)—H(12) | 119.0 | |
| C(1)—C(2)—Cl(1) | 124.3 | (17) | C(13)—C(12)—H(12) | 119.0 | |
| C(2)—C(3)—C(4) | 121.4 | (16) | C(8)—C(13)—C(12) | 114.5 | (4) |
| C(2)—C(3)—H(3) | 119.3 | | C(8)—C(13)—C(14) | 123.9 | (3) |
| C(4)—C(3)—H(3) | 119.3 | | C(12)—C(13)—C(14) | 121.6 | (5) |
| C(5)—C(4)—C(3) | 119.4 | (13) | C(13)—C(14)—C(18) | 112.0 | (4) |
| C(13)—C(14)—C(15) | 113.8 | (4) | N(3)—C(20)—C(19) | 124.6 | (3) |
| C(18)—C(14)—C(15) | 109.1 | (3) | N(4)—C(20)—C(19) | 122.6 | (3) |
| C(13)—C(14)—H(14) | 107.2 | | N(4)—C(21)—C(22) | 113.6 | (2) |
| C(18)—C(14)—H(14) | 107.2 | | N(4)—C(21)—H(21A) | 108.8 | |
| C(15)—C(14)—H(14) | 107.2 | | C(22)—C(21)—H(21A) | 108.8 | |
| C(16)—C(15)—C(14) | 111.6 | (3) | N(4)—C(21)—H(21B) | 108.8 | |
| C(16)—C(15)—H(15A) | 109.3 | | C(22)—C(21)—H(21B) | 108.8 | |
| C(14)—C(15)—H(15A) | 109.3 | | H(21A)—C(21)—H(21B) | 107.7 | |
| C(16)—C(15)—H(15B) | 109.3 | | O(3)—C(22)—C(21) | 113.4 | (3) |
| C(14)—C(15)—H(15B) | 109.3 | | O(3)—C(22)—C(23) | 91.1 | (4) |
| H(15A)—C(15)—H(15B) | 108.0 | | C(21)—C(22)—C(23) | 115.1 | (3) |
| N(2)—C(16)—C(15) | 110.8 | (3) | O(3)—C(22)—H(22) | 111.9 | |
| N(2)—C(16)—H(16A) | 109.5 | | C(21)—C(22)—H(22) | 111.9 | |
| C(15)—C(16)—H(16A) | 109.5 | | C(23)—C(22)—H(22) | 111.9 | |
| N(2)—C(16)—H(16B) | 109.5 | | C(24)—C(23)—C(22) | 86.1 | (4) |
| C(15)—C(16)—H(16B) | 109.5 | | C(24)—C(23)—H(23A) | 114.3 | |
| H(16A)—C(16)—H(16B) | 108.1 | | C(22)—C(23)—H(23A) | 114.3 | |
| N(2)—C(17)—C(18) | 110.9 | (3) | C(24)—C(23)—H(23B) | 114.3 | |
| N(2)—C(17)—H(17A) | 109.5 | | C(22)—C(23)—H(23B) | 114.3 | |
| C(18)—C(17)—H(17A) | 109.5 | | H(23A)—C(23)—H(23B) | 111.5 | |
| N(2)—C(17)—H(17B) | 109.5 | | O(3)—C(24)—C(23) | 93.6 | (4) |
| C(18)—C(17)—H(17B) | 109.5 | | O(3)—C(24)—H(24A) | 113.0 | |
| H(17A)—C(17)—H(17B) | 108.1 | | C(23)—C(24)—H(24A) | 113.0 | |
| C(17)—C(18)—C(14) | 111.0 | (3) | O(3)—C(24)—H(24B) | 113.0 | |
| C(17)—C(18)—H(18A) | 109.4 | | C(23)—C(24)—H(24B) | 113.0 | |
| C(14)—C(18)—H(18A) | 109.4 | | H(24A)—C(24)—H(24B) | 110.4 | |
| C(17)—C(18)—H(18B) | 109.4 | | C(30)—C(25)—N(4) | 131.7 | (2) |
| C(14)—C(18)—H(18B) | 109.4 | | C(30)—C(25)—C(26) | 122.5 | (2) |
| H(18A)—C(18)—H(18B) | 108.0 | | N(4)—C(25)—C(26) | 105.8 | (2) |
| N(2)—C(19)—C(20) | 112.5 | (2) | N(3)—C(26)—C(25) | 109.5 | (2) |
| N(2)—C(19)—H(19A) | 109.1 | | N(3)—C(26)—C(27) | 131.0 | (2) |
| C(20)—C(19)—H(19A) | 109.1 | | C(25)—C(26)—C(27) | 119.6 | (2) |
| N(2)—C(19)—H(19B) | 109.1 | | C(28)—C(27)—C(26) | 118.3 | (2) |
| C(20)—C(19)—H(19B) | 109.1 | | C(28)—C(27)—H(27) | 120.9 | |
| H(19A)—C(19)—H(19B) | 107.8 | | C(26)—C(27)—H(27) | 120.9 | |
| N(3)—C(20)—N(4) | 112.7 | (2) | C(27)—C(28)—C(29) | 121.6 | (3) |

| C(27)—C(28)—H(28) | 119.2 | |
|---|---|---|
| C(29)—C(28)—H(28) | 119.2 | |
| C(30)—C(29)—C(28) | 120.4 | (2) |
| C(30)—C(29)—C(31) | 119.7 | (2) |
| C(28)—C(29)—C(31) | 119.8 | (2) |
| C(29)—C(30)—C(25) | 117.7 | (2) |
| C(29)—C(30)—H(30) | 121.2 | |
| C(25)—C(30)—H(30) | 121.2 | |
| O(5)—C(31)—O(4) | 125.1 | (3) |
| O(5)—C(31)—C(29) | 118.7 | (2) |
| O(4)—C(31)—C(29) | 116.2 | (2) |
| N(5)—C(32)—C(34) | 109.1 | (2) |
| N(5)—C(32)—C(33) | 106.4 | (2) |
| C(34)—C(32)—C(33) | 111.4 | (2) |
| N(5)—C(32)—C(35) | 108.6 | (2) |
| C(34)—C(32)—C(35) | 109.8 | (2) |
| C(33)—C(32)—C(35) | 111.5 | (2) |
| O(6)—C(33)—C(32) | 110.1 | (2) |
| O(6)—C(33)—H(33A) | 109.6 | |
| C(32)—C(33)—H(33A) | 109.6 | |
| O(6)—C(33)—H(33B) | 109.6 | |
| C(32)—C(33)—H(33B) | 109.6 | |
| H(33A)—C(33)—H(33B) | 108.2 | |
| O(7)—C(34)—C(32) | 110.8 | (2) |
| O(7)—C(34)—H(34A) | 109.5 | |
| C(32)—C(34)—H(34A) | 109.5 | |
| O(7)—C(34)—H(34B) | 109.5 | |
| C(32)—C(34)—H(34B) | 109.5 | |
| H(34A)—C(34)—H(34B) | 108.1 | |
| O(8)—C(35)—C(32) | 113.0 | (2) |

| | |
|---|---|
| O(8)—C(35)—H(35A) | 109.0 |
| C(32)—C(35)—H(35A) | 109.0 |
| O(8)—C(35)—H(35B) | 109.0 |
| C(32)—C(35)—H(35B) | 109.0 |
| H(35A)—C(35)—H(35B) | 107.8 |

Symmetry transformations used to generate equivalent atoms:

TABLE E3-4
Anisotropic displacement parameters (Å² × 10³) for Form 3.
The anisotropic displacement factor exponent takes the
form: −2π²[h² a*²U¹¹ + . . . + 2 h k a* b* U¹²]

| | U¹¹ | U²² | U³³ | U²³ | U¹³ | U¹² |
|---|---|---|---|---|---|---|
| Cl(1) | 121(2) | 543(11) | 312(4) | −88(7) | 38(2) | 21(4) |
| N(1) | 123(5) | 171(6) | 147(5) | 38(5) | −24(4) | −23(5) |
| N(2) | 56(1) | 44(1) | 49(1) | 0(1) | 9(1) | −3(1) |
| N(3) | 38(1) | 55(1) | 51(1) | −4(1) | 1(1) | −8(1) |
| N(4) | 38(1) | 48(1) | 47(1) | −1(1) | 2(1) | −2(1) |
| N(5) | 38(1) | 50(1) | 54(1) | −6(1) | 2(1) | 1(1) |
| O(1) | 160(4) | 152(4) | 77(2) | 20(2) | −31(2) | −19(4) |
| O(2) | 101(2) | 141(3) | 70(2) | 29(2) | −13(2) | −25(2) |
| O(3) | 100(2) | 84(2) | 89(2) | −4(2) | −30(2) | −16(2) |
| O(4) | 37(1) | 59(1) | 79(1) | 6(1) | 1(1) | −7(1) |
| O(5) | 45(1) | 62(1) | 71(1) | 19(1) | 2(1) | −5(1) |
| O(6) | 38(1) | 53(1) | 77(1) | −11(1) | 2(1) | −7(1) |
| O(7) | 61(1) | 48(1) | 99(2) | 13(1) | 14(1) | 2(1) |
| O(8) | 42(1) | 46(1) | 85(1) | −6(1) | −4(1) | 3(1) |
| O(1W) | 48(1) | 54(1) | 72(1) | 3(1) | 1(1) | −2(1) |
| C(1) | 113(6) | 286(17) | 154(7) | 53(10) | −9(5) | −33(9) |
| C(2) | 95(6) | 277(19) | 196(10) | −10(11) | −40(6) | 5(9) |
| C(3) | 156(10) | 233(17) | 296(18) | −36(15) | −1(11) | 77(12) |
| C(4) | 162(8) | 142(8) | 258(12) | 43(9) | 36(8) | 6(7) |
| C(5) | 112(4) | 126(5) | 98(3) | 30(3) | −33(3) | −22(4) |
| C(6) | 114(4) | 120(4) | 85(3) | 17(3) | −22(3) | −12(3) |
| C(7) | 167(7) | 114(5) | 167(7) | 21(5) | −30(5) | −7(5) |
| C(8) | 107(3) | 98(3) | 55(2) | 17(2) | 5(2) | 6(3) |
| C(9) | 140(5) | 110(4) | 62(2) | 9(2) | −8(2) | 2(4) |
| C(10) | 172(6) | 147(6) | 60(2) | 18(3) | 0(3) | 1(5) |
| C(11) | 180(7) | 148(6) | 68(3) | 32(3) | 31(3) | −4(6) |
| C(12) | 126(4) | 127(4) | 71(2) | 30(3) | 24(3) | −4(4) |
| C(13) | 96(3) | 90(3) | 59(2) | 14(2) | 15(2) | 4(2) |
| C(14) | 79(2) | 83(2) | 59(2) | 15(2) | 14(2) | −5(2) |
| C(15) | 92(3) | 76(2) | 53(2) | 0(2) | 20(2) | 23(2) |
| C(16) | 68(2) | 65(2) | 60(2) | 7(2) | 14(1) | 16(2) |
| C(17) | 86(2) | 45(2) | 72(2) | 2(2) | 1(2) | 2(2) |
| C(18) | 98(3) | 55(2) | 73(2) | 10(2) | 0(2) | 12(2) |
| C(19) | 54(2) | 51(2) | 57(1) | −3(1) | 3(1) | −10(1) |
| C(20) | 44(1) | 50(1) | 45(1) | −5(1) | 5(1) | −7(1) |
| C(21) | 40(1) | 56(2) | 52(1) | 2(1) | 4(1) | 5(1) |
| C(22) | 50(2) | 78(2) | 53(2) | 1(2) | −1(1) | 1(2) |
| C(23) | 64(2) | 132(5) | 85(3) | 5(3) | −22(2) | 11(3) |
| C(24) | 79(3) | 170(7) | 134(4) | 16(5) | −36(3) | −38(4) |
| C(25) | 36(1) | 50(1) | 40(1) | −2(1) | 1(1) | 0(1) |
| C(26) | 35(1) | 52(1) | 41(1) | −8(1) | 2(1) | −7(1) |
| C(27) | 31(1) | 63(2) | 44(1) | −4(1) | −4(1) | −3(1) |
| C(28) | 40(1) | 58(2) | 38(1) | 3(1) | −2(1) | 1(1) |
| C(29) | 36(1) | 52(1) | 38(1) | −3(1) | 2(1) | −4(1) |
| C(30) | 31(1) | 53(1) | 44(1) | 1(1) | 0(1) | −3(1) |
| C(31) | 36(1) | 53(2) | 45(1) | −2(1) | 3(1) | −3(1) |
| C(32) | 35(1) | 44(1) | 51(1) | −4(1) | −1(1) | −2(1) |
| C(33) | 39(1) | 52(2) | 62(2) | 0(1) | 6(1) | −3(1) |
| C(34) | 43(1) | 50(2) | 68(2) | 0(1) | 1(1) | 2(1) |
| C(35) | 38(1) | 50(2) | 58(1) | −7(1) | 4(1) | −1(1) |

Acquisition of Powder X-Ray Diffraction (PXRD) Data for Form 3 of Compound 1, 1,3-dihydroxy-2-(hydroxymethyl)propan-2-aminium salt (Also Known as Form 3 of Monohydrate of Tris Salt of Compound 1)

A sample of Form 3 (e.g., the white solid of the tris salt of Compound 1 prepared according to the method described herein) was submitted for PXRD analysis and found to be a crystalline material (which is designated as Form 3).

Powder X-ray diffraction analysis was conducted using a Bruker AXS D8 Endeavor diffractometer equipped with a Cu radiation source (K-α average). The divergence slit was set at 15 mm continuous illumination. Diffracted radiation was detected by a PSD-Lynx Eye detector, with the detector PSD opening set at 2.99 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.00999 degrees and a step time of 1.0 second. The antiscatter screen was set to a fixed distance of 1.5 mm. Samples were rotated at 15/min during collection. Samples were prepared by placing them in a silicon low background sample holder and rotated during collection. Data were collected using Bruker DIFFRAC Plus software and analysis was performed by EVA diffract plus software. The sample holder used in a particular experiment is given by a codename within the filename: DW=Deep well holder, SD=small divot holder and FP=Flat plate holder.

The PXRD data file was not processed prior to peak searching. Using the peak search algorithm in the EVA software, peaks selected with a threshold value of 1 and a width value of 0.3 were used to make preliminary peak assignments. The output of automated assignments was visually checked to ensure validity and adjustments were manually made if necessary. Peaks with relative intensity of 3% were generally chosen. The peaks which were not resolved or were consistent with noise were not selected. A typical error associated with the peak position from PXRD stated in USP up to +/−0.2° 2-Theta (USP-941). A list of PXRD diffraction peaks expressed in terms of the degree 26 and relative intensities with a relative intensity of 3.0% from a sample of Form 3 is provided below.

TABLE E3-5
PXRD Peaks and Relative Intensities of Form 3

| Degrees 2⊖ | |
|---|---|
| (Angle) ± 0.2° 2⊖ | Relative Intensity |
| 3.7* | 16% |
| 7.4* | 45% |
| 7.7 | 28% |
| 7.9 | 10% |
| 9.9* | 11% |
| 10.2 | 7% |
| 11.1* | 7% |
| 12.8 | 3% |
| 14.1 | 16% |
| 14.3 | 20% |
| 14.8* | 49% |
| 15.8 | 12% |
| 16.1 | 27% |
| 16.6 | 14% |
| 17.4 | 48% |
| 18.2* | 12% |
| 18.6 | 4% |
| 19.6 | 42% |
| 19.9* | 100% |
| 20.0 | 98% |
| 20.6* | 36% |
| 21.6 | 18% |
| 23.1 | 18% |
| 23.5* | 17% |
| 24.3* | 39% |
| 24.6* | 25% |
| 25.9 | 5% |
| 26.1 | 5% |
| 26.6 | 12% |
| 27.0 | 11% |
| 27.3 | 8% |
| 27.7 | 14% |
| 28.9 | 7% |
| 30.5 | 5% |
| 30.9 | 15% |
| 31.6 | 8% |
| 34.2 | 8% |
| 35.2 | 4% |
| 35.9 | 5% |
| 37.2 | 7% |

### Solid State NMR Analysis of Form 3 of Tris Salt of Compound 1(Monohydrate)

Solid state NMR (ssNMR) analysis was conducted on a CPMAS probe positioned into a Bruker-BioSpin Avance III 500 MHz (¹H frequency) NMR spectrometer. A sample of Form 3 of 1,3-Dihydroxy-2-(hydroxymethyl)propan-2-aminium Salt of Compound 1, monohydrate was packed into a 4 mm rotor. A magic angle spinning rate of 15.0 kHz was used.

¹³C ssNMR spectrum was collected using a proton decoupled cross-polarization magic angle spinning (CPMAS) experiment. A phase modulated proton decoupling field of 80-90 kHz was applied during spectral acquisition. The cross-polarization contact time was set to 2 ms and the recycle delay of 3-8 seconds. The number of scans was adjusted to obtain an adequate signal to noise ratio, with 2048 scans being collected for each API. The ¹³C chemical shift scale was referenced using a ¹³C CPMAS experiment on an external standard of crystalline adamantane, setting its up-field resonance to 29.5 ppm.

Automatic peak picking was performed using Bruker-BioSpin TopSpin version 3.6 software. Generally, a threshold value of 3% relative intensity was used for preliminary peak selection. The output of the automated peak picking was visually checked to ensure validity and adjustments were manually made if necessary. Although specific solid state NMR peak values are reported herein there does exist a range for these peak values due to differences in instruments, samples, and sample preparation. This is common practice in the art of solid state NMR because of the variation inherent in peak positions. A typical variability for a ¹³C chemical shift x-axis value is on the order of plus or minus 0.2 ppm for a crystalline solid. The solid state NMR peak heights reported herein are relative intensities. Solid state NMR intensities can vary depending on the actual setup of the CPMAS experimental parameters and the thermal history of the sample. The chemical shift data is dependent on the testing conditions (i.e. spinning speed and sample holder), reference material, and data processing parameters, among other factors. Typically, the ss-NMR results are accurate to within about ±0.2 ppm.

TABLE E3-6
Carbon chemical shifts observed (Characteristic peaks are starred).

| ¹³C Chemical Shifts [ppm] ± 0.2 ppm | Relative Intensity |
|---|---|
| 21.0 | 75 |
| 24.2 | 67 |
| 29.9 | 71 |
| 32.1 | 67 |
| 42.8* | 85 |
| 51.9 | 59 |
| 52.8 | 72 |
| 54.7* | 100 |
| 59.3 | 62 |
| 61.2 | 66 |
| 62.3 | 44 |
| 68.2 | 72 |
| 83.2 | 87 |
| 106.0 | 67 |
| 114.8 | 41 |
| 116.2 | 65 |
| 119.9 | 56 |
| 122.4 | 99 |
| 122.7 | 88 |
| 123.8 | 80 |
| 125.5 | 58 |
| 128.2* | 83 |
| 132.3 | 58 |
| 133.5 | 43 |
| 137.2 | 45 |
| 138.4* | 63 |
| 142.8 | 55 |
| 144.3 | 54 |
| 147.2 | 79 |
| 147.5 | 89 |
| 153.7 | 58 |
| 156.6* | 46 |
| 172.6 | 44 |

### Example AA. CHO GLP-1R Clone H6—Assay 1

GLP-1R-mediated agonist activity was determined with a cell-based functional assay utilizing an HTRF (Homogeneous Time-Resolved Fluorescence) cAMP detection kit (cAMP HI Range Assay Kit; CisBio cat #62AM6PEJ) that measures cAMP levels in the cell. The method is a competitive immunoassay between native cAMP produced by the cells and exogenous cAMP labeled with the dye d2. The tracer binding is visualized by a mAb anti-cAMP labeled with Cryptate. The specific signal (i.e. energy transfer) is inversely proportional to the concentration of cAMP in either standard or experimental sample.

The human GLP-1R coding sequence (NCBI Reference Sequence NP_002053.3, including naturally-occurring variant Gly168Ser) was subcloned into pcDNA3 (Invitrogen) and a cell line stably expressing the receptor was isolated (designated Clone H6). Saturation binding analyses (filtration assay procedure) using ¹²⁵I-GLP-1₇₋₃₆ (Perkin Elmer) showed that plasma membranes derived from this cell line express a high GLP-1R density (K_{d}: 0.4 nM, Bₘₐₓ: 1900 fmol/mg protein).

Cells were removed from cryopreservation, re-suspended in 40 mL of Dulbecco's Phosphate Buffered Saline (DPBS—Lonza Cat #17-512Q) and centrifuged at 800×g for 5 minutes at 22° C. The cell pellet was then re-suspended in 10 mL of growth medium [DMEM/F12 1:1 Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081) and 500 μg/mL Geneticin (G418) (Invitrogen #10131035)]. A 1 mL sample of the cell suspension in growth media was counted on a Becton Dickinson ViCell to determine cell viability and cell count per mL. The remaining cell suspension was then adjusted with growth media to deliver 2000 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment in 5% carbon dioxide.

Varying concentrations of each compound to be tested (in DMSO) were diluted in assay buffer (HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E) containing 100 μM 3-isobutyl-1-methylxanthin (IBMX; Sigma cat #15879). The final DMSO concentration is 1%.

After 48 hours, the growth media was removed from the assay plate wells, and the cells were treated with 20 μL of the serially diluted compound in assay buffer for 30 minutes at 37° C. in a humidified environment in 5% carbon dioxide. Following the 30 minute incubation, 10 μL of labeled d2 cAMP and 10 μL of anti-cAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM cAMP by interpolation from a cAMP standard curve (as described in the manufacturer's assay protocol) and the percent effect was determined relative to a saturating concentration of the full agonist GLP-1₇₋₃₆ (1 μM) included on each plate. EC₅₀ determinations were made from agonist dose-response curves analyzed with a curve fitting program using a 4-parameter logistic dose response equation.

### Example BB. CHO GLP-1R Clone C6—Assay 2

GLP-1R-mediated agonist activity was determined with a cell-based functional assay utilizing an HTRF (Homogeneous Time-Resolved Fluorescence) cAMP detection kit (cAMP HI Range Assay Kit; Cis Bio cat #62AM6PEJ) that measures cAMP levels in the cell. The method is a competitive immunoassay between native cAMP produced by the cells and exogenous cAMP labeled with the dye d2. The tracer binding is visualized by a mAb anti-cAMP labeled with Cryptate. The specific signal (i.e. energy transfer) is inversely proportional to the concentration of cAMP in either a standard or an experimental sample.

The human GLP-1R coding sequence (NCBI Reference Sequence NP_002053.3, including naturally-occurring variant Leu260Phe) was subcloned into pcDNA5-FRT-TO and a clonal CHO cell line stably expressing a low receptor density was isolated using the Flp-In™ T-Rex™ System, as described by the manufacturer (ThermoFisher). Saturation binding analyses (filtration assay procedure) using ¹²⁵I-GLP-1 (Perkin Elmer) showed that plasma membranes derived from this cell line (designated clone C6) express a low GLP-1R density (K_{d}: 0.3 nM, Bₘₐₓ: 240 fmol/mg protein), relative to the clone H6 cell line.

Cells were removed from cryopreservation, re-suspended in 40 mL of Dulbecco's Phosphate Buffered Saline (DPBS—Lonza Cat #17-512Q) and centrifuged at 800×g for 5 minutes at 22° C. The DPBS was aspirated, and the cell pellet was re-suspended in 10 mL of complete growth medium (DMEM:F12 1:1Mixture with HEPES, L-Gln, 500 mL (DMEM/F12 Lonza Cat #12-719F), 10% heat inactivated fetal bovine serum (Gibco Cat #16140-071), 5 mL of 100× Pen-Strep (Gibco Cat #15140-122), 5 mL of 100× L-Glutamine (Gibco Cat #25030-081), 700 μg/mL Hygromycin (Invitrogen Cat #10687010) and 15 μg/mL Blasticidin (Gibco Cat #R21001). A 1 mL sample of the cell suspension in growth media was counted on a Becton Dickinson ViCell to determine cell viability and cell count per mL. The remaining cell suspension was then adjusted with growth media to deliver 1600 viable cells per well using a Matrix Combi Multidrop reagent dispenser, and the cells were dispensed into a white 384 well tissue culture treated assay plate (Corning 3570). The assay plate was then incubated for 48 hours at 37° C. in a humidified environment (95% O₂, 5% CO₂)

Varying concentrations of each compound to be tested (in DMSO) were diluted in assay buffer [HBSS with Calcium/Magnesium (Lonza/BioWhittaker cat #10-527F)/0.1% BSA (Sigma Aldrich cat #A7409-1L)/20 mM HEPES (Lonza/BioWhittaker cat #17-737E)] containing 100 μM 3-isobutyl-1-methylxanthin (IBMX; Sigma cat #15879). The final DMSO concentration in the compound/assay buffer mixture is 1%.

After 48 hours, the growth media was removed from the assay plate wells, and the cells were treated with 20 μL of the serially diluted compound in assay buffer for 30 minutes at 37° C. in a humidified environment (95% O₂, 5% CO₂). Following the 30 minute incubation, 10 μL of labeled d2 cAMP and 10 μL of anti-cAMP antibody (both diluted 1:20 in cell lysis buffer; as described in the manufacturer's assay protocol) were added to each well of the assay plate. The plates were then incubated at room temperature and after 60 minutes, changes in the HTRF signal were read with an Envision 2104 multi-label plate reader using excitation of 330 nm and emissions of 615 and 665 nm. Raw data were converted to nM cAMP by interpolation from a cAMP standard curve (as described in the manufacturer's assay protocol) and the percent effect was determined relative to a saturating concentration of the full agonist GLP-1 (1 μM) included on each plate. EC₅₀ determinations were made from agonist dose response curves analyzed with a curve fitting program using a 4-parameter logistic dose response equation.

In Table X-1, assay data are presented to two (2) significant figures as the geometric mean (EC₅₀s) and arithmetic mean (Emax) based on the number of replicates listed (Number). A blank cell means there was no data for that Example or the Emax was not calculated.

TABLE X-1
Biological activity for Compound 1.

| | Assay 1 | Assay 1 | | Assay 2 | Assay 2 | |
|---|---|---|---|---|---|---|
| | EC₅₀ | Emax | Assay 1 | EC₅₀ | Emax | Assay 2 |
| Compound | (nM) | (%) | Number | (nM) | (%) | Number |
| Compound | 0.96 | 99 | 5 | 17 | 96 | 8 |
| 1**** | | | | | | |

****Tested as formate salt and free acid

All patents, patent applications and references referred to herein are hereby incorporated by reference in their entirety.

1. A hydrate crystalline form of 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, 1,3-dihydroxy-2-(hydroxymethyl)propan-2-amine salt.

2. The hydrate crystalline form of claim 1 wherein the hydrate crystalline form is a monohydrate crystalline form.

3. The hydrate crystalline form of claim 2, wherein the crystalline form is Form 2, and wherein Form 2 has a powder X-ray diffraction pattern (PXRD) comprising at least two peaks, in terms of 2θ, at 7.1±0.2°, 7.6±0.2°, 10.7±0.2°, and 19.4±0.2°.

4. The monohydrate crystalline form of claim 3, wherein Form 2 has a powder X-ray diffraction pattern (PXRD) comprising at least three peaks, in terms of 2θ, at 7.1±0.2°, 7.6±0.2°, 10.7±0.2°, and 19.4±0.2°.

5. The monohydrate crystalline form of claim 4, wherein Form 2 has a powder X-ray diffraction pattern (PXRD) comprising peaks, in terms of 2θ, at 7.1±0.2°, 7.6±0.2°, 10.7±0.2°, and 19.4±0.2°.

6. A monohydrate crystalline form (Form 3) of 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, 1,3-dihydroxy-2-(hydroxymethyl)propan-2-amine salt, wherein Form 3 has a PXRD comprising at least two peaks, in terms of 2θ, at 3.7±0.2°, 7.4±0.2°, 9.9±0.2°, 14.8±0.2°, and 20.6±10.2°.

7. The monohydrate crystalline form of claim 6, wherein the PXRD comprises peaks, in terms of 2θ, at 3.7±0.2°, 7.4±0.2°, and 14.8±0.2°.

8. The monohydrate crystalline form of claim 6, wherein the PXRD comprises peaks, in terms of 2θ, at 3.7±0.2°, 7.4±0.2°, 14.8±0.2°, and 20.6±0.2°.

9. The monohydrate crystalline form of claim 6, wherein the PXRD comprises peaks, in terms of 2θ, at 3.7±10.2°, 7.4±0.2°, 9.9±0.2°, 14.8±0.2°, and 20.6±0.2°.

10. The monohydrate crystalline form of claim 6, wherein the monohydrate crystalline form has a ¹³C ssNMR spectrum comprising chemical shifts at 54.7±0.2 ppm and 138.4±0.2 ppm.

11. The monohydrate crystalline form of claim 10, wherein the ¹³C ssNMR spectrum comprises chemical shifts at 54.7±0.2 ppm, 138.4±0.2 ppm, and 156.6 ppm±0.2 ppm.

12. The monohydrate crystalline form of claim 10, wherein the ¹³C ssNMR spectrum comprises chemical shifts at 42.8±0.2 ppm, 54.7±0.2 ppm, 128.2±0.2 ppm, 138.4±0.2 ppm, and 156.6±0.2 ppm.

13. The hydrate crystalline form of claim 1, wherein said hydrate crystalline form is substantially pure.

14. A pharmaceutical composition comprising a therapeutically effective amount of the hydrate crystalline form of claim 1 and a pharmaceutically acceptable carrier.

15. A pharmaceutical composition comprising a therapeutically effective amount of the hydrate crystalline form of claim 2 and a pharmaceutically acceptable carrier.

16. A pharmaceutical composition comprising a therapeutically effective amount of the hydrate crystalline form of claim 3 and a pharmaceutically acceptable carrier.

17. A pharmaceutical composition comprising a therapeutically effective amount of the monohydrate crystalline form of claim 6 and a pharmaceutically acceptable carrier.

18-21. (canceled)

22. An amorphous form of 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, 1,3-dihydroxy-2-(hydroxymethyl)propan-2-amine salt.

23. The amorphous form of claim 22, wherein said amorphous form is substantially pure.

24. A pharmaceutical composition comprising a therapeutically effective amount of an amorphous form of claim 22 and a pharmaceutically acceptable carrier.

25. (canceled)

26. A pharmaceutical composition comprising a therapeutically effective amount of 2-((4-((S)-2-(5-chloropyridin-2-yl)-2-methylbenzo[d][1,3]dioxol-4-yl)piperidin-1-yl)methyl)-1-(((S)-oxetan-2-yl)methyl)-1H-benzo[d]imidazole-6-carboxylic acid, 1,3-dihydroxy-2-(hydroxymethyl)propan-2-amine salt (“tris salt of Compound 1”) and a pharmaceutically acceptable carrier, wherein the tris salt of Compound 1 comprises a crystalline form of tris salt of Compound 1 and an amorphous form of tris salt of Compound 1.

27. (canceled)

28. A method for treating a disease or disorder in a human in need of such treatment comprising administering to the human a therapeutically effective amount of a hydrate crystalline form of any one of claims 1, 2, 3, and 6, wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

29. A method for treating a disease or disorder in a human in need of such treatment comprising administering to the human a therapeutically effective amount of an amorphous form of claim 22, wherein the disease or disorder is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, NASH with fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, Polycystic Ovary Syndrome, and addiction.

30-31. (canceled)

32. The method of claim 28, wherein the disease or disorder is selected from obesity, NAFLD, NASH, NASH with fibrosis, T2D, and a cardiovascular disease.

33. The method of claim 29, wherein the disease or disorder is selected from obesity, NAFLD, NASH, NASH with fibrosis, T2D, and a cardiovascular disease.
